(19) United States
(12) Patent Application Publication
(10) Pub. No.: US 2012/0251448 A1
(43) Pub. Date: Oct. 4, 2012

(54) Compounds for Use in the Detection of Neurodegenerative Diseases

(76) Inventors: Franz F. Hefti, Bernardsville, NJ (US); Geoffrey Golding, Philadelphia, PA (US); Ximin Li, Ardmore, PA (US); Seok-Rye Choi, Aston, PA (US); Luke Esposito, Seattle, WA (US); Marisa-Claire Yadon, Seattle, WA (US); Joel Cummings, Seattle, WA (US); F. Michael Hudson, Seattle, WA (US); Thomas Lake, Snohomish, WA (US); Alan D. Snow, Lynnwood, WA (US)

(21) Appl. No.: 13/411,403

(22) Filed: Mar. 2, 2012

Related U.S. Application Data
(60) Provisional application No. 61/448,740, filed on Mar. 3, 2011.

(51) Int. Cl. A61K 51/04; A61K 49/00

(52) U.S. Cl. 424/1.89; 424/9.6; 424/9.1; 424/1.65; 424/9.3; 424/1.81; 424/1.85

(57) ABSTRACT

Provided are the use of the disclosed compounds as imaging agents and methods for in vivo imaging and detection of pathological characteristics unique to synuclein diseases, such as Parkinson's disease.

### CROSS REFERENCE TO RELATED APPLICATIONS

This application claims priority to U.S. Provisional Patent Application No. 61/448,740, filed Mar. 3, 2011, the disclosure of which is hereby incorporated by reference in its entirety for all purposes.

### TECHNICAL FIELD

Provided herein are the use of compounds and compositions for imaging agents and methods for the detection of neurodegenerative diseases such as Parkinson's disease.

### BACKGROUND OF INVENTION

Parkinson's disease (PD) is a neurodegenerative human disorder characterized clinically by both motor (movement) and non-motor behavioral dysfunction, and histopathologically by the formation, deposition, accumulation and/or persistence of abnormal fibrillar protein deposits. This accumulation of cytoplasmic Lewy bodies consisting of fibrils/aggregates of α-synuclein/NAC (non-Aβ component) is believed important in the pathogenesis of PD. Lewy bodies occur mostly in the substantia nigra and locus ceruleus sections of the brain stem and the olfactory bulb, but also, to a lesser extent, in other subcortical and cortical regions of the brain. Because of this specific localization in the brain, Lewy bodies interfere with the health and integrity of dopaminergic neuronal projections from the substantia nigra to the striatum, thus adversely affecting the ability to initiate, carry out and control voluntary movements. Lewy bodies present in these brain regions may also impact the production of acetylcholine and/or the balance between dopamine and acetylcholine in the brain, thus causing disruption in perception, thinking and behavior as well as other non-motor symptoms including loss of smell, constipation, and sleep disorders.

Dementia with Lewy Bodies (DLB) is a progressive neurodegenerative disorder characterized by symptoms which display various degrees of manifestation. Such symptoms include progressive dementia, Parkinsonian movement difficulties, hallucinations, and increased sensitivity to neuroleptic drugs. As with Alzheimer's disease (AD), advanced age is considered to be a risk factor for DLB, with average onset typically between the ages of 50-85.20% of all dementia cases are caused by DLB and over 50% of PD patients develop Parkinson's Disease Dementia (PDD), a type of DLB. DLB may occur alone or in conjunction with other brain abnormalities, including those involved in AD and PD, as mentioned above. Currently, conclusive diagnosis of DLB is made during postmortem autopsy.

There has been a wide interest in developing radiolabeled ligands that bind to and allow in vivo imaging of α-synuclein and Lewy bodies. In Alzheimer's disease, another neurodegerative disease, several radioisotopically-labeled Aβ-aggregate specific ligands have been reported for the imaging of amyloid plaque in the living subject using positron emission tomography (PET) or single photon emission computed tomography (SPECT). These ligands are mainly targeted to nigrostriatal neurons and D2/D3 receptors in the brain. Examples of such radioisotopically-labeled Aβ-aggregate specific ligands include; ^{99m}Tc-TRODAT-1, ¹²³I-IBZM, and ¹¹C-Pittsburgh compound B (PIB) among others. In addition, several radiopharmaceuticals have been used for PET or SPECT imaging of regional cerebral perfusion such as ¹⁵O-labeled water (H₂¹⁵O) and ¹³N-ammonia (¹³NH₃). SPECT radiopharmaceuticals such as Tc-^{99m} HMPAO and Tc-^{99m} Bicisate are also used as cerebral perfusion agents.

Dual-isotope imaging techniques have been employed in trials where simultaneous ¹⁸F-FDG and ^{99m}Tc-HMPAO SPECT imaging technique was utilized to image selected areas in the neuroanatomy involved in anxiety and depression such as the hippocampus, basal ganglia and gyri temporales superiores. There have also been studies employing a dual SPECT imaging technique with ^{99m}Tc-TRODAT-I and ¹²³I-IBZM to image nigrostriatal neurons and D2/D3 receptors.

New agents or compounds able to bind with high specificity to α-synuclein and/or NAC fibrils and/or aggregates are regarded as potential imaging agents to aid in detection of diseases and are much sought after.

Structural imaging of the brain with CT or MRI rarely leads to a definitive diagnosis in parkinsonian patients. Functional radio-isotope imaging techniques provide more definitive detection and diagnosis. Imaging the binding of ligands to dopamine transporters on presynaptic nigrostriatal neurons, SPECT can help differentiate parkinsonian syndromes from syndromes where there is no such degeneration. Scans are graded visually depending on the severity of cell loss. The scans of patients with parkinsonism associated with presynaptic neuronal degeneration should be abnormal with lower ligand binding and, therefore, display less bright uptake on the scan. Typically, in early unilateral PD there is selective loss of nigrostriatal neurons to the putamen on the side contralateral to the clinically affected limb (grade 1 scan). With progression, the degeneration affects both putamen (grade 2) when the clinical signs will often be bilateral before going on to affect the caudate nuclei as well (grade 3), again often with lower uptake contralateral to the clinically worse side. Vascular disease can give atypical scan appearances which do not fit with this pattern (for example, focal loss of uptake in one caudate but not the putamen due to focal infarction). Patients with non-parkinsonian syndromes (such as essential tremor) or parkinsonism where there is no presynaptic neuronal degeneration (for example, drug induced) should have normal scans.

Although there is growing consensus that FP-CIT SPECT can be helpful in certain clinical situations, there are few high quality studies that give reliable data on its sensitivity and specificity. Reported sensitivities (87-98%) and specificities (80-100%) are high—but not perfect—for differentiating between parkinsonism with presynaptic degeneration and non-degenerative syndromes. Clearly better imaging agents are needed to target the pathological indicators of Parkinson's disease and aid in detection and diagnosis.

### Parkinson's Disease and Synucleinopathies

Parkinson's disease is a neurodegenerative disorder that is pathologically characterized by the presence of intracytoplasmic Lewy bodies (Lewy in Handbuch der Neurologie, M. Lewandowski, ed., Springer, Berlin, pp. 920-933, 1912; Pollanen et al., J. Neuropath. Exp. Neurol. 52:183-191, 1993), the major components of which are filaments consisting of α-synuclein (Spillantini et al., Proc. Natl. Acad. Sci. USA 95:6469-6473, 1998; Arai et al., Neurosci. Lett. 259:83-86, 1999), a 140-amino acid protein (Ueda et al., Proc. Natl. Acad. Sci. USA 90:11282-11286, 1993). Three dominant mutations in α-synuclein causing increased tendency to aggregate and resulting in familial early onset Parkinson's disease have been described suggesting that Lewy bodies contribute mechanistically to the degeneration of neurons in Parkinson's disease and related disorders (Polymeropoulos et al., Science 276:2045-2047, 1997; Kruger et al., Nature Genet. 18:106-108, 1998; Zarranz et al., Ann. Neurol. 55:164-173, 2004). Recently, in vitro studies have demonstrated that recombinant α-synuclein can indeed form Lewy body-like fibrils (Conway et al., Nature Med. 4:1318-1320, 1998; Hashimoto et al., Brain Res. 799:301-306, 1998; Nahri et al., J. Biol. Chem. 274:9843-9846, 1999; Choi et al., FEBS Lett. 576:363-368, 2004). Most importantly, both the A53T and the E46K Parkinson's disease-linked α-synuclein mutations accelerate this fibril-forming aggregation process, demonstrating that such in vitro studies may have relevance for Parkinson's disease pathogenesis. Alphα-synuclein aggregation and fibril formation fulfills the criteria of a nucleation-dependent polymerization process (Wood et al., J. Biol. Chem. 274:19509-19512, 1999). Alphα-synuclein recombinant protein, and non-Aβ component (known as NAC), which is a 35-amino acid peptide fragment of α-synuclein, both have the ability to form fibrils when incubated at 37° C., and are positive with stains such as Congo red (demonstrating a red/green birefringence when viewed under polarized light) and Thioflavin S (demonstrating positive fluorescence) (Hashimoto et al., Brain Res. 799:301-306, 1998; Ueda et al., Proc. Natl. Acad. Sci. USA 90:11282-11286, 1993).

Synucleins are a family of small, presynaptic neuronal proteins composed of α-, β-, and γ-synucleins, of which only α-synuclein aggregates have been associated with several neurological diseases (Ian et al., Clinical Neurosc. Res. 1:445-455, 2001; Trojanowski and Lee, Neurotoxicology 23:457-460, 2002). The role of synucleins (and in particular, α-synuclein) in the etiology of a number of neurodegenerative diseases has developed from several observations. Pathologically, synuclein was identified as a major component of Lewy bodies, the hallmark inclusions of Parkinson's disease, and a fragment thereof was isolated from amyloid plaques of a different neurological disease, Alzheimer's disease. Biochemically, recombinant α-synuclein was shown to form fibrils that recapitulated the ultrastructural features of α-synuclein isolated from patients with dementia with Lewy bodies, Parkinson's disease and multiple system atrophy. Additionally, the identification of mutations within the α-synuclein gene, albeit in rare cases of familial Parkinson's disease, demonstrated an unequivocal link between synuclein pathology and neurodegenerative diseases. The common involvement of α-synuclein in a spectrum of diseases such as Parkinson's disease, dementia with Lewy bodies, multiple system atrophy and the Lewy body variant of Alzheimer's disease has led to the classification of these diseases under the umbrella term of “synucleinopathies”.

The compounds shown herein could serve as imaging agents useful for the detection and diagnosis of Parkinson's disease and other synucleinopathies.

### SUMMARY OF INVENTION

In a first aspect, provided herein are the use of compounds as imaging agents for detecting in vivo aggregation of α-synuclein.

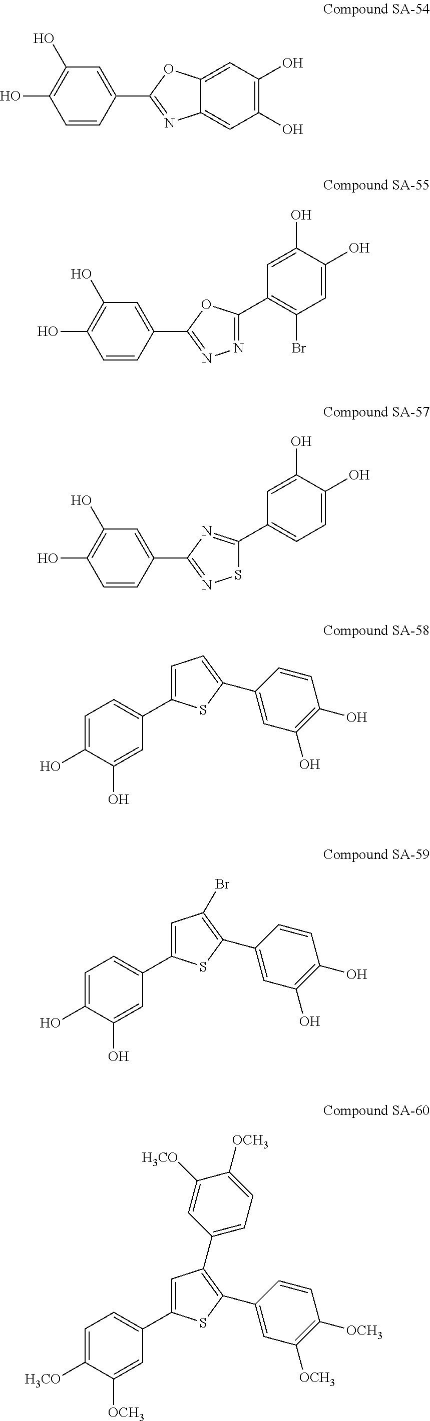

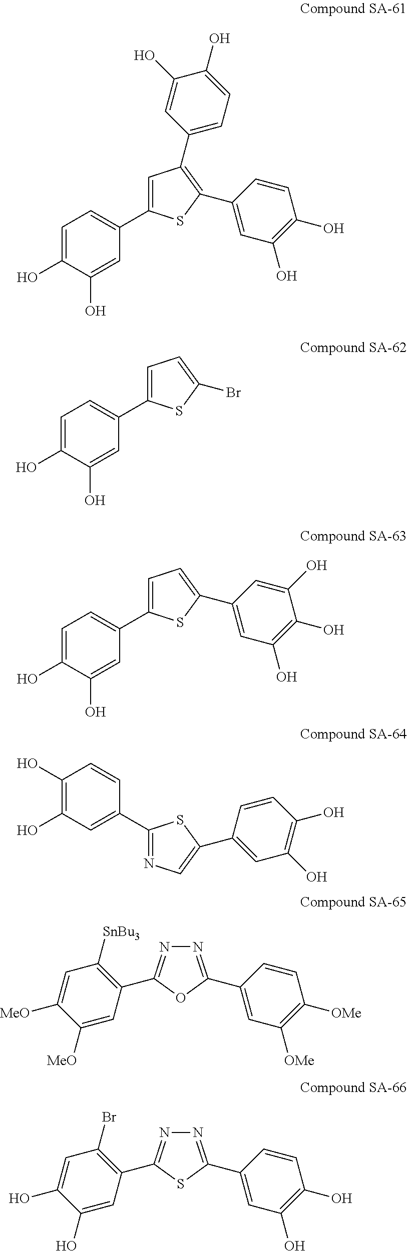

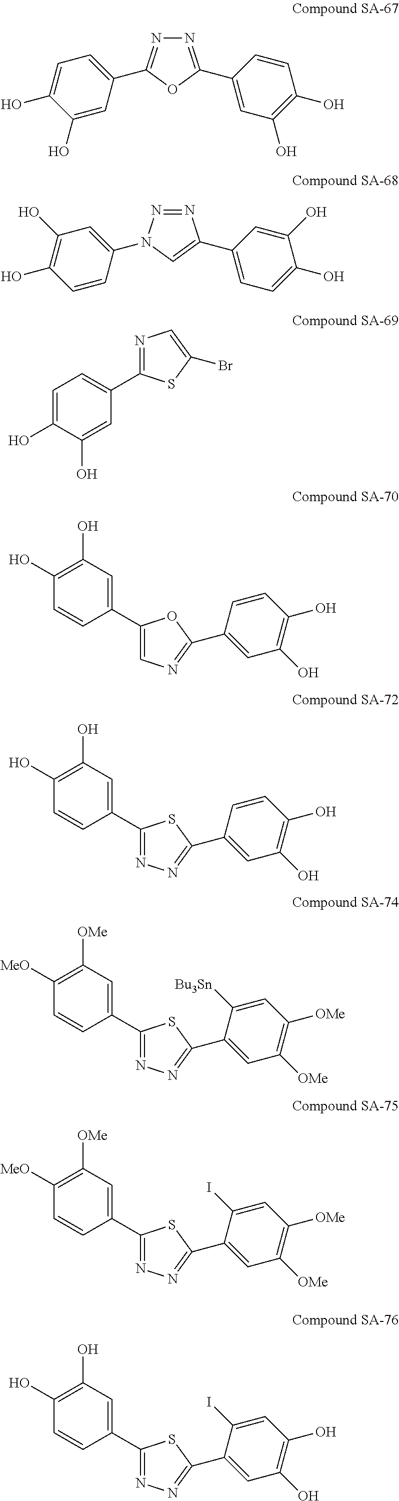

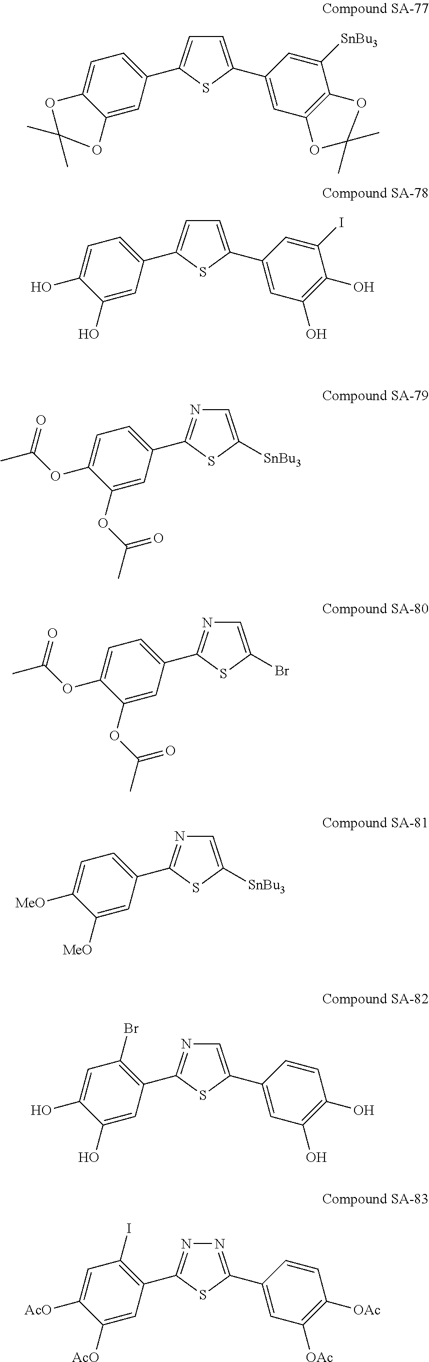

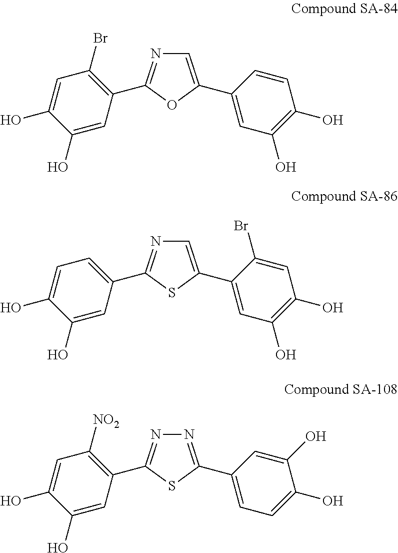

In a second aspect, provided herein is a method of in vivo imaging of α-synuclein aggregates, comprising introducing into a mammal a detectable amount of a compound selected from

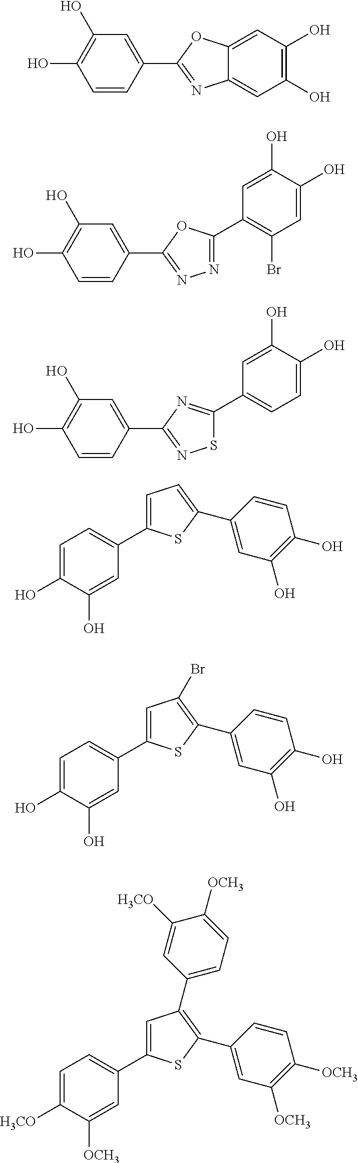

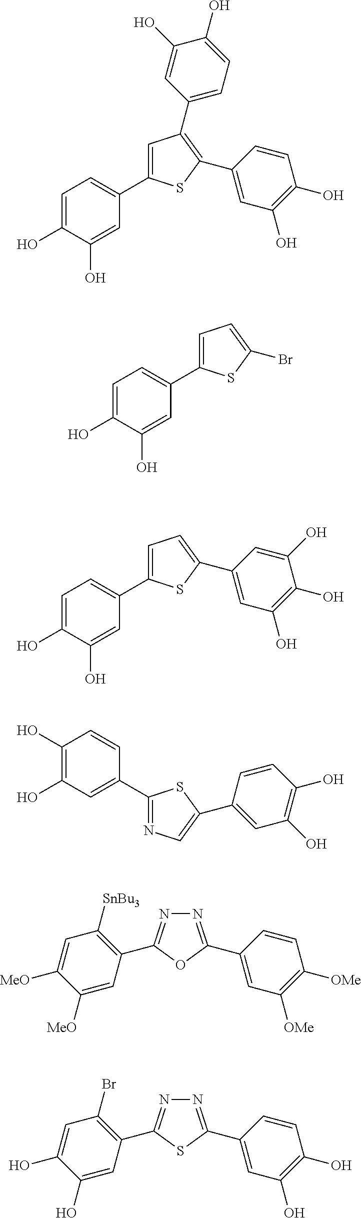

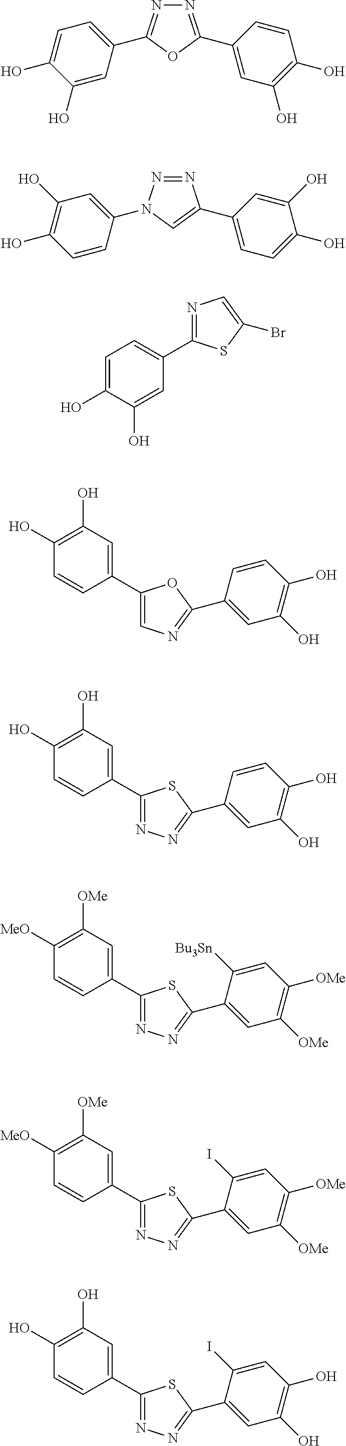

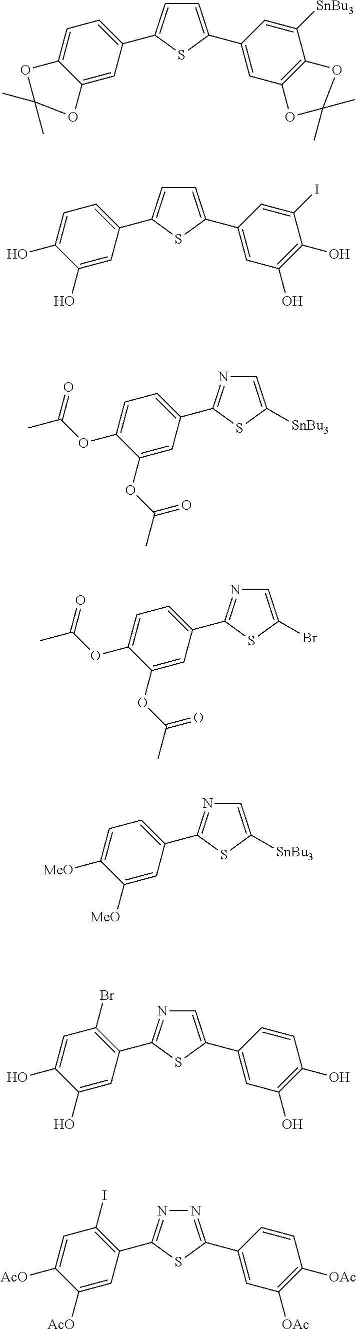

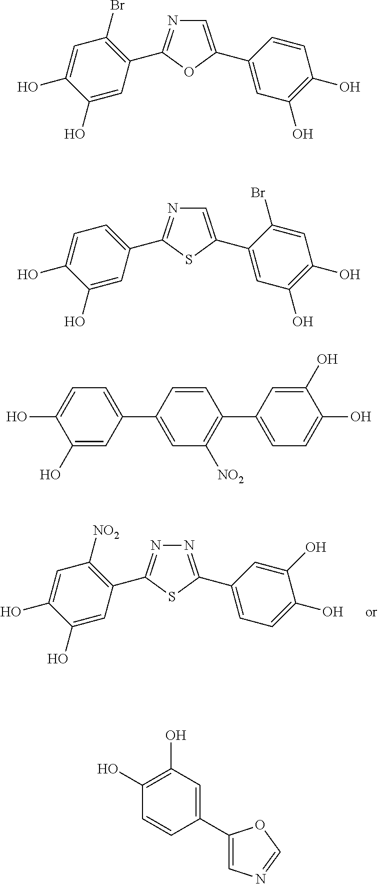

which has been labeled with a detectable substance, allowing a sufficient time period for the labeled compound to become associated with α-synuclein aggregates, and detecting the labeled compound associated with one or more α-synuclein aggregates.

In some embodiments of this aspect, the method includes the additional step of quantifying the amount of labeled compound associated with the one or more α-synuclein aggregates.

In other embodiments of this aspect, the step of detecting the labeled compound is by positron emission tomography or single photon emission computed tomography.

In further embodiments of this aspect, the detectable substance is selected from a group including radioisotopes, phosphorescent compounds, fluorescent compounds, fluorescent proteins, paramagnetic compounds, metal chelators, and enzymes.

In some embodiments of this aspect, the radioisotope can be isotopes of Br, ¹²³I, ¹²⁵I, ¹³¹I, ^{99m}Tc, ¹¹C and ¹⁸F.

In yet further embodiments of this aspect, the sufficient time period for the labeled compound to become associated with α-synuclein aggregates is from about 5 minutes to a time corresponding to approximately twice the radioactive half-life of the radioisotope of the labeled compound, or is from about 10 minutes to a time corresponding to the radioactive half-life of the radioisotope of the labeled compound.

In other embodiments of this aspect, the sufficient time period for the labeled compound to become associated with α-synuclein aggregates is about 1 to about 60 minutes.

In further embodiments of this aspect, the detectable amount of labeled compound comprises from about 0.1 to about 20 mCi.

In yet further embodiments of this aspect, the labeled compound is in dosage form for intravenous injection.

In a third aspect, provided herein is a kit for detecting α-synuclein aggregates in the brain of an individual, containing: a labeled compound; and instructions for using the labeled compound, where the instructions contain a direction to administer to an individual a detectable amount of labeled compound; a direction to wait a sufficient period of time; and a direction to detect the labeled compound.

### DETAILED DESCRIPTION OF THE INVENTION

### A. Definitions

Unless defined otherwise, all technical and scientific terms used herein have the same meaning as is commonly understood by one of ordinary skill in the art to which this invention belongs. All patents, applications, published applications and other publications are incorporated by reference in their entirety. In the event that there are a plurality of definitions for a term herein, those in this section prevail unless stated otherwise.

As used herein, “Synuclein diseases” or “synucleinopathies” are diseases associated with the formation, deposition, accumulation, or persistence of synuclein fibrils and/or aggregates, including, but not limited to α-synuclein fibrils. Such diseases include, but are not limited to Parkinson's disease, Familial Parkinson's disease, Lewy body disease, the Lewy body variant of Alzheimer's disease, dementia with Lewy bodies, multiple system atrophy, and the Parkinsonism-dementia complex of Guam.

Aggregation or Fibrillogenesis refers to the formation, deposition, accumulation and/or persistence of synuclein fibrils, filaments, inclusions, deposits, and/or NAC fibrils, filaments, inclusions, deposits, and/or aggregates or the like.

“Mammal” includes both humans and non-human mammals, such as companion animals (cats, dogs, and the like), laboratory animals (such as mice, rats, guinea pigs, and the like) and farm animals (cattle, horses, sheep, goats, swine, and the like).

As used herein, “NAC” (non-Aβ component) is a 35-amino acid peptide fragment of α-synuclein, which like α-synuclein, has the ability to form fibrils when incubated at 37° C., and is positive with stains such as Congo red (demonstrating a red/green birefringence when viewed under polarized light) and Thioflavin S (demonstrating positive fluorescence) (Hashimoto et al., Brain Res. 799:301-306, 1998; Ueda et al., Proc. Natl. Acad. Sci. U.S.A. 90:11282-11286, 1993).

### B. Imaging Agents and Scans

Methods for detecting the presence or absence of α-synuclein aggregates in a biological sample are provided. These methods include contacting a biological sample with a selected compound, wherein the compound is labeled with a detectable substance, for example, with a radionucleotide, phosphorescent compound, fluorescent compound, fluorescent protein, paramagnetic compound, metal chelators, or enzyme, all of which are readily detectable in various assays and diagnostics known to those skilled in the art, and then detecting the detectable substance bound to the α-synuclein aggregates in the biological sample.

Methods for imaging the presence or absence of α-synuclein aggregates in the body or biological tissues are provided. These methods include contacting α-synuclein aggregates in the body with a compound, wherein the compound is labeled with detectable substance, for example, with a radionucleotide, phosphorescent compound, fluorescent compound, fluorescent protein, paramagnetic compound, metal chelator, or enzyme, and detecting the detectable substance bound to the α-synuclein aggregates in the body or biological tissues.

Accordingly, the compounds can also be used as imaging agents to detect the presence or absence of α-synuclein aggregates in a biological sample or in vivo in a subject. Furthermore, detection of α-synuclein aggregates using the compounds can be used to help diagnose synucleopathies in a subject. For non-limiting examples of the use of imaging agents see U.S. patent application number 2009/0257949 published Oct. 15, 2009, the contents of which are incorporated herein by reference.

Compounds which interact with α-synuclein or derivatives thereof are also disclosed herein. The compounds can be used for the detection applications as described herein.

In another embodiment, a compound is used in vivo to detect, and if desired, quantitate, α-synuclein deposition in a subject, for example, to aid in the diagnosis of synucleopathies in the subject. To aid in detection, the compound can be modified with a detectable substance, preferably a radioisotope.

Methods for labeling compounds with technetium and other radioisotopes are known in the art. A modifying group can be chosen that provides a site at which a chelation group for ^{99m}Tc can be introduced, such as a derivative of cholic acid, which has a free amino group. Also provided are compounds labeled with radioactive fluorine. For example the isotope of radioactive fluorine can be incorporated to create a diagnostic agent. Preferably using ¹⁸F for positron emission tomography (PET) or single photon emission computed tomography (SPECT) studies.

Radiolabeled compounds can be prepared using known methods (Choi, S, et al., J. Nucl. Med 50(11):1887-94, 2009). Briefly, ¹⁸F-fluoride ion trapped on an anion exchange cartridge (ORTG, Inc.) is eluted to the reaction vessel using 1 mL of aqueous solution consisting of potassium carbonate (50 mg) and Kryptofix (100 mg) in a mixture of H₂O (4 mL) and MeCN (10 mL). The water in the eluted activities is removed by heating to 70° C. under a stream of helium and under vacuum, and this process is repeated to yield anhydrous Kryptofix (Sigma Chemicals)/K₂CO₃/18F-fluoride. Approximately 1 mg of O-tosylated precursor for making ¹⁸F-labeled compounds is dissolved in 1 mL of anhydrous dimethyl sulfoxide and added to the reactor mixture containing the anhydrous ¹⁸F-fluoride prepared above. The reaction mixture is heated to 120° C. for 10 min and then cooled to 50° C. before the addition of 0.25 mL of 10% HCl in high-performance liquid chromatography (HPLC)-grade water. The mixture is heated to 120° C. for 5 min. After cooling to 50° C., a 0.33-mL portion of 10% NaOH and 5-mL HPLC-grade water is added. The solution is loaded onto a Sep-Pak Light C18 column (Waters) and washed further with 5 mL of HPLC water; the desired ¹⁸F-compound is eluted with 1 mL of MeCN into a reservoir containing 2 mL of HPLC solvent (55% MeCN:45% 20 mM aqueous NH₄OAc with 0.5% w/v ascorbic acid sodium salt) and 1 mL of HPLC water.

The crude mixture is then loaded onto a semipreparative HPLC column (Eclipse XDB-C18; Zorbax) (5 μm, 9.4×250 mm; flow rate, 4 mL/min). The HPLC fraction containing the desired ¹⁸F-compound is collected into another reservoir containing 15 mL of HPLC water and passed through a preconditioned Sep-Pak Light C18, which is further washed with 15 mL of sterile water for an injection containing 0.5% w/v ascorbic acid sodium salt. The final product, is eluted with 1 mL of ethanol (United States Pharmacopeia [USP] for injection) into 9 mL of sodium chloride injection (sterile, USP) containing 0.5% w/v ascorbic acid sodium salt (USP). ¹⁸F-compounds can be routinely prepared with a 10%-30% radiochemical yield, a high specific activity (>37,000-185,000 MBq/mmol [>1,000-5,000 Ci/mmol]), and a high radiochemical purity (>99%).

The techniques and methodology that use positron emission tomography to study the brain are known being first conceived in the 1950's. For example there is a current NIMH sponsored PET study (NCT00024622) of regional cerebral dopamine neurochemistry and blood flow in normal volunteers and patients with Parkinson's disease (both familial and sporadic). Using PET with 6-[¹⁸F] Fluoro-L-dopa (FDOPA) and ¹⁵O—H₂O in a single scan session, both presynaptic dopaminergic function and regional cerebral blood flow (rCBF) are to be assessed. The kinetic rate constant (Ki) for presynaptic dopaminergic uptake in striatum and other regions is calculated. Cerebral findings are also related to allelic variation in genes of interest. Comparisons between subjects with inherited vs. sporadic Parkinson's disease will be conducted to determine whether the PET phenotype is the same in both groups. Each subject is further screened with an MRI to rule out structural abnormalities and also to further delineate areas of interest in the PET scans.

The following non-limiting Examples are given by way of illustration only and are not considered a limitation of the subject matter, many apparent variations of which are possible without departing from the spirit or scope thereof.

### EXAMPLES

### Example 1

### Synthesis of SA-54

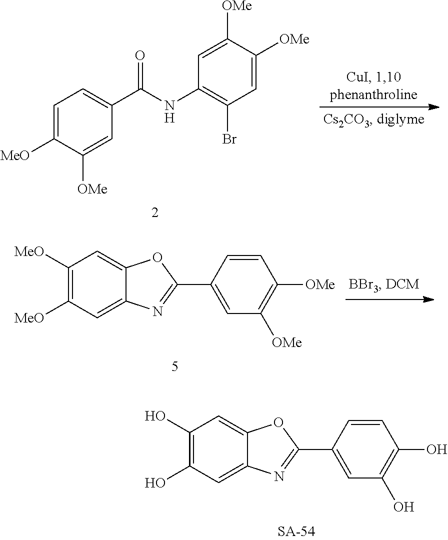

To 0.230 g (0.58 mmol) of benzamide 2 was added 0.017 g of CuI (0.09 mmol), 0.031 g (0.17 mmol) of 1,10 phenanthroline, and 0.560 g (1.7 mmol) of Cs₂CO₃. The mixture was suspended in 5 mL of diglyme, and heated to 140° C. for 20 h. The mixture was diluted with 30 mL DCM, washed three times with 20 mL of water, dried (Na₂SO₄), and concentrated to give an orange oil as the crude product. This was heated under vacuum (1 mm Hg) until a light orange solid formed to give 0.135 g (75% yield) of benzoxazole 5, which was used without further purification. ¹H NMR (200 MHz, CDCl₃) δ 7.74 (dd, J=2.0 Hz, 8.4 Hz, 1H), 7.67 (d, J=2.2 Hz, 1H), 7.21 (s, 3H), 7.10 (s, 3H), 6.95 (d, J=8.4 Hz, 1H), 3.98-3.89 (4 overlapping singlets, 12H).

To 0.071 g (0.23 mmol) of 5 in 5 mL of DCM at 0° C. was added 2 mL 1M BBr₃ in DCM. The dark brown mixture was stirred 6 h, quenched with 5 mL MeOH, and concentrated. The MeOH dilution-concentration procedure was repeated three more times to give 0.081 g of crude product. This product was purified by PTLC using 5% MeOH/DCM followed by 10% MeOH/DCM as the eluent and gave 0.023 g (39% yield) of SA-54 as an off-white solid. ¹H NMR (200 MHz) δ 8.84 (bs, 2H), 8.34 (bs, 1H), 7.35 (bs, 1H), 7.47 (d, J=2 Hz, 1H), 7.40 (dd, J=2 Hz, 8.2 Hz, 1H), 7.05 (s, 2H), 7.01 (s, 2H). HRMS Calculated for C₁₃H₁₀NO₅ (M+H)⁺ 260.0586 found 260.0559.

### Example 2

### Synthesis of SA-55 and SA-65

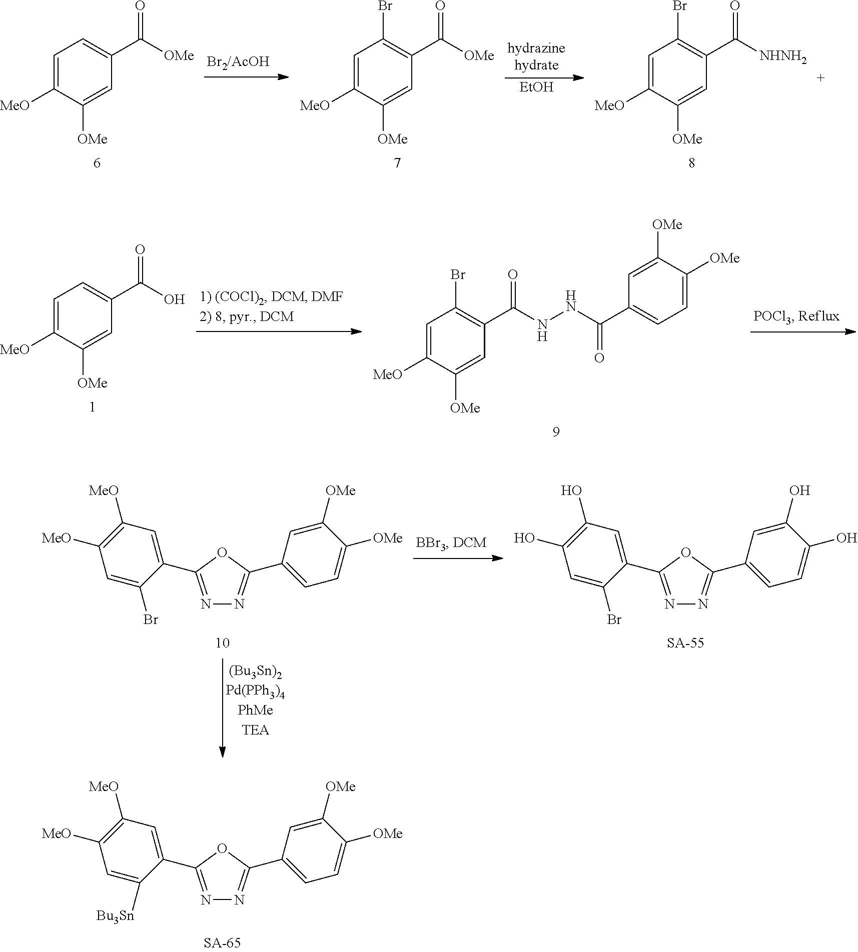

To 10.00 g (51 mmol) of methyl 3,4 dimethoxybenzoate (6) in 50 mL of AcOH at 0° C. was added 8.90 g (56 mmol) of Br₂ in 50 mL of AcOH over 1.5 h. The ice bath was removed and the mixture stirred 45 min. The reaction was quenched by pouring into 700 mL of H₂O, stirred 30 min, left quiescent for 1 h, and filtered. The collected solid was washed with H₂O and washed with sat. aq. Na₂S₂O₃. The solid was partially dried, dissolved in 300 mL hot MeOH, and the resultant solution was cooled. The cool methanolic solution of product was treated with 200 mL of H₂O and the white solid filtered to give 8.92 g (64% yield) of methyl-2-bromo-4,5-dimethoxybenzoate (7) as a white powder. The compound matched the physical and spectral properties of the known compound.

A mixture of 0.960 g (3.48 mmol) of 7, 1.60 g (35 mmol) of hydrazine hydrate (62% hydrazine), and 5 mL of EtOH was refluxed for 15 h. The mixture was cooled to −20° C., vacuum filtered, washed with 50 mL of ice-cold 1:1 EtOH:H₂O, and dried to give 0.832 g (87% yield) of (2-bromo-4,5-dimethoxy benzoyloxy) hydrazine (8) as a white, needle-like crystalline solid. The above procedure was repeated on 2.79 g of the starting ester 7 to result in 2.77 g (99% yield) of 8. ¹H NMR (200 MHz, CDCl₃) δ 6.97 (s, 1H), 6.86 (s, 1H), 3.85 (s, 6H). HRMS Calculated for C₉H₁₂O₃N₂Br 275.0031. found 275.0037.

To 2.15 g (11.8 mmol) of 3,4-dimethoxybenzoic acid (1) in 10 mL of DCM was added sequentially 2.5 mL (29.1 mmol) oxalyl chloride and 0.2 mL of DMF. The mixture was stirred for 16 h during which time it became a clear, light yellow solution. This solution was concentrated and dried thoroughly to remove the excess oxalyl chloride to generate the crude acid chloride as a light yellow solid. This solid was taken up in 20 mL of DCM, the solution cooled to 0° C., and treated with 10 mL of pyridine and 0.25 g of DMAP. The resultant solution was treated with 1.69 g (6.15 mmol) of 8 in 10 mL DCM and 10 mL of pyridine. The mixture was stirred 3 h at 0° C. and warmed to 23° C. The reaction was stirred an additional 16 h, concentrated, taken up in 50 mL of EtOAc, and the layers separated. The aqueous was extracted once more with 50 mL EtOAc. The combined organic layers were washed three times with 100 mL H₂O, dried (Na₂SO₄), and concentrated. The concentrate was purified by Flash 40+M column chromatography (Biotage) eluting first with 150 mL of 1:1 EtOAc/Hex and then 2 L 5:1 EtOAc/Hex to give 1.27 g (47% yield) of hydrazide 9 as a yellow-brown powder. The reaction was repeated using 2.20 g of 3,4 dihydroxybenzoic acid (1) and 2.77 g of hydrazine 8 to give 2.60 g (49% yield) of hydrazide 9. ¹H NMR (200 MHz, CDCl₃) δ 9.91 (d, J=5.2 Hz, 1H), 9.53 (d, J=5.0 Hz, 1H), 7.46 (dd, J=2.0 Hz, 8.4 Hz, 1H), 7.41 (d, J=2.2 Hz, 1H), 7.23 (s, 1H), 6.98 (s, 1H), 6.80 (d, J=8.4 Hz, 1H), 3.96-3.89 (4 overlapping singlets, 12H).

A solution of 0.352 g of intermediate 9 in 3 mL of POCl₃ was refluxed for 3 h. The reaction mixture is cooled, poured into 125 mL of water, and sonicated for one minute. The suspension was allowed to stand for 1 h, and the solid was filtered, washed with excess water, collected, and air-dried to give 0.302 g (90% yield) of brominated tetramethoxyoxadiazole (10) as a white solid. ¹H NMR (200 MHz, CDCl₃) δ 7.71 (dd, J=2.0 Hz, 8.4 Hz, 1H), 7.67 (d, J=2.0 Hz, 1H), 7.57 (s, 1H), 7.17 (s, 1H), 6.98 (d, J=8.2 Hz, 1H), 3.96 (4 overlapping singlets, 12H). HRMS Calculated for C₁₄H₁₀BrN₂O₅ (M+H)⁺ 421.0339. found 421.0334.

A mixture of 0.038 g of intermediate 10 in 3 mL of DCM was cooled to −78° C., and treated dropwise with a solution of 0.450 g of BBr₃ in 5 mL of DCM. The mixture was stirred at −78° C. for 1 h, then at 23° C. for 2.5 h. The mixture was quenched by adding it carefully to 5 mL of MeOH in a 100 mL flask. The methanol solution was concentrated to 1 mL, diluted with 5 mL water, and filtered to give 0.017 g (56% yield) of SA-55 as a light yellow solid. ¹H NMR (200 MHz, CDCl₃) δ 10.5-9.5 (overlapping broad singlets, 4H), 7.56 (d, J=2 Hz, 1H), 7.52 (s, 1H), 7.48 (dd, J=2 Hz, 8.6 Hz, 1H), 7.25 (s, 1H), 7.04 (d, J=8.4 Hz, 1H)

### Example 3

### Synthesis of SA-57

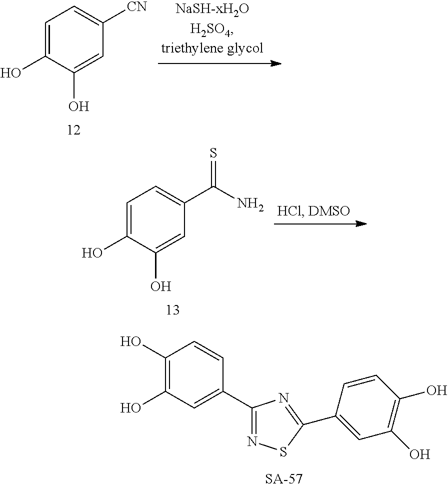

To a thick-walled 15 mL tube with a resealable Teflon screw-cap was added 0.584 g (4.32 mmol) of 3,4-dihydroxbenzonitrile (12), 5 mL of triethylene glycol, 1.172 g of NaSH.xH₂O, and 0.25 mL of concentrated H₂SO₄. The tube was sealed with the cap, and the mixture was warmed to 110° C. and stirred for 3 days at this temperature. The reaction was quenched by pouring into 100 mL sat. aq. NH₄⁺Cl, and extracted twice with 50 mL of EtOAc. The combined organics were washed three times with 15 mL water, dried with NaSO₄, and concentrated to give 0.512 g (71% yield) of 13 as a golden colored solid. ¹H NMR (200 MHz, CDCl₃) δ 9.67 (s, 1H), 9.29 (s, 1H), 7.56 (dd, J=1.8 Hz, 8.2 Hz), 7.49 (d, J=1.8 Hz), 6.94 (d, J=8.2 Hz), 6.09 (s, 2H). ¹³C NMR (50 MHz, CDCl₃) δ 198.9, 150.5, 147.4, 133.7, 123.3, 108.2, 107.8, 102.3.

A solution of 0.40 g (2.37 mmol) of 13 in 7 mL of DMSO was treated with 12 drops of concentrated HCl, warmed to 38° C. for 18 h, and poured into 25 mL of brine. The resultant solid was filtered and washed with water to give 0.21 g (59% yield) of SA-57 as a yellow solid. ¹H-NMR (DMSO-d6) 9.85 (bs, 1H) 9.52 (bs, 1H), 9.43 (bs, 1H), 9.28 (bs, 1H), 7.69 (d, 1H, J=2 Hz), 7.59 (dd, 1H, J=2.2, 8.2 Hz), 7.46 (d, 1H, J=2 Hz), 7.38 (dd, 1H, J=2.2, 8.2 Hz), 6.90 (d, 1H, J=8.2 Hz), 6.86 (d, 1H, J=8.4 Hz). ¹³C-NMR (DMSO-d6) 187.8, 173.4, 150.2, 148.5, 146.4, 145.9, 124.6, 122.0, 120.4, 120.2, 116.8, 116.2, 115.7, 114.5. HRMS-ESI Calculated for C₁₄H₁₁N₂O₄S (M+H)⁺ 303.0440. found 303.0448.

### Example 4

### Synthesis of SA-58

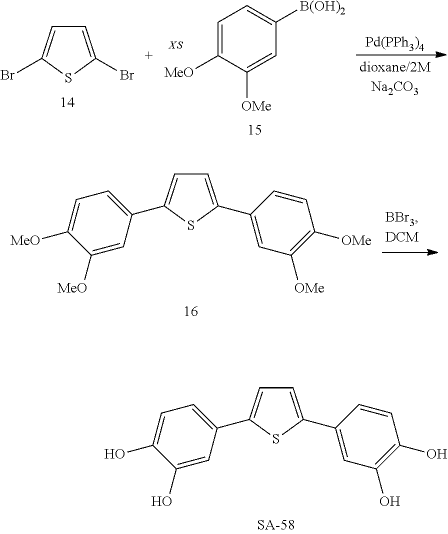

To a solution of 2,5-dibromothiophene (14) (242 mg, 1 mmol), (3,4-dimethoxy phenyl)boronic acid (15) (455 mg, 2.5 mmol), and Pd(PPh₃)₄ (58 mg, 0.05 mmol) in dioxane (10 mL) was added Na₂CO₃ (12 mL, 2.0 M aqueous solution). The resultant mixture was purged with nitrogen and stirred rapidly while heating at 90° C. overnight. The reaction mixture was cooled to 23° C., acidified with 1M HCl and extracted with EtOAc. The combined organic extracts were washed with H₂O, dried over MgSO₄, filtered and concentrated under reduced pressure. 2,5-bis(3′,4′-dimethoxyphenyl)thiophene (16) was obtained quantitatively as a green-yellow solid after the purification by column chromatography (10%-20% EtOAc in hexanes).

To a solution of 2,5-bis(3′,4′-dimethoxyphenyl)thiophene 16 (110 mg, 0.3 mmol) in dry dichloromethane at −78° C. was added BBr₃ (3 mL, 1M solution in DCM, 2.5 equiv per methoxy function) dropwise. The reaction mixture was stirred at −78° C. for 3 h, warmed to 23° C., and stirred 16 h under nitrogen atmosphere. Water (10 mL) was added to quench the reaction, and the aqueous layer was extracted with EtOAc. The combined organic layer was washed with brine, dried over MgSO₄, filtered, and concentrated under reduced pressure. The product was purified by recrystallization in MeOH/DCM and SA-58 was obtained quantitatively as a greenish solid.

### Example 5

### Synthesis of SA-59, SA-60 and SA-61

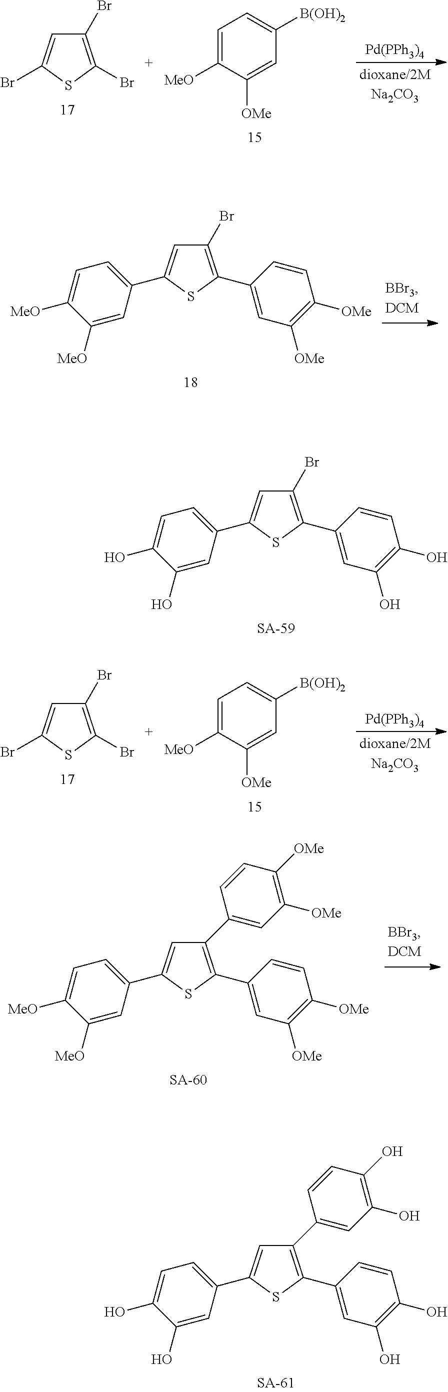

3-bromo-2,5-bis(3′,4′-dimethoxyphenyl)thiophene (18) was prepared by the reaction of 2,3,5-tribromothiophene (17) (321 mg, 1 mmol) and (3,4-dimethoxyphenyl)boronic acid (15) (419 mg, 2.3 mmol) according to the similar procedure for compound 16. The reaction mixture was purified by column chromatography (10%-30% EtOAc in hexanes) and afforded 18 (337 mg, 77% yield) as a yellow solid. SA-60 was also isolated (97 mg, 20% yield) from the reaction above as a dark yellow solid.

SA-59 was prepared by the reaction of 3-bromo-2,5-bis(3′,4′-dimethoxyphenyl)thiophene (18) (258 mg, 0.59 mmol) and BBr₃ (6 mL, 6 mmol) according to the similar procedure for compound SA-58. SA-59 (157 mg, 70% yield) was obtained after preparative thin layer chromatography (PTLC) purification (10% MeOH in DCM) as a green solid.

SA-61 was prepared by the reaction of 2,3,5-tri(3′,4′-dimethoxyphenyl)thiophene (SA-60) (68 mg, 0.14 mmol) and BBr₃ (2 mL, 2 mmol) according to the similar procedure for compound SA-58. SA-61 (34 mg, 60% yield) was obtained after PTLC purification (10% MeOH in DCM) as a brown oil.

### Example 6

### Synthesis of SA-62

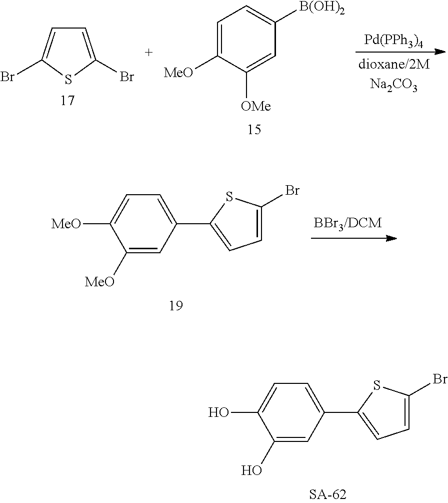

Compound 19 was prepared by the reaction of 2,5-dibromothiophene (17) (1.14 g, 5.24 mmol) and (3,4-dimethoxyphenyl)boronic acid (15) (910 mg, 5 mmol) according to the similar procedure for compound 16. The reaction mixture was purified by flash column chromatography (FCC) (5%-20% EtOAc in hexanes) and afforded compound 19 (509 mg, 34% yield) as a yellowish crystal. Compound 16 was also isolated (578 mg, 65% yield) as a yellow solid.

SA-62 was prepared by the reaction of 2-bromo-5-(3,4-dimethoxyphenyl)thiophene (19) (60 mg, 0.2 mmol) and BBr₃ (1M in DCM, 1 mL, 1 mmol) and isolated as a green solid in quantitative yield.

### Example 7

### Synthesis of SA-63

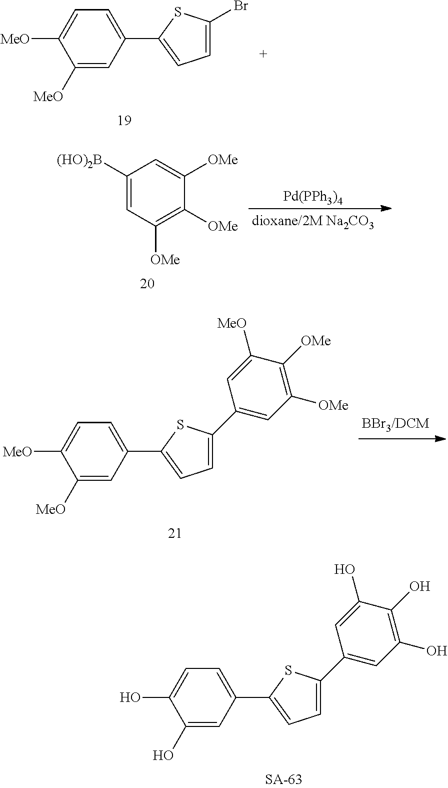

Compound 21 was prepared by the reaction of 2-bromo-5-(3,4-dimethoxyphenyl)thiophene (19) (449 mg, 1.5 mmol) and (3,4,5-trimethoxyphenyl)boronic acid (20) (382 mg, 1.8 mmol) according to the similar procedure for compound 16 and purified by column chromatography (10%-20% EtOAc in hexanes), then recrystallization (EtOAc) provided the desired compound as a yellow solid (524 mg, 91% yield).

SA-63 was prepared by the reaction of 2-(3,4-dimethoxyphenyl)-5-(3,4,5-trimethoxyphenyl)thiophene (21) (47 mg, 0.12 mmol) and BBr₃ (1M in DCM, 0.8 mL, 0.8 mmol). SA-63 was obtained quantitatively as dark blue solid.

### Example 8

### Synthesis of SA-64

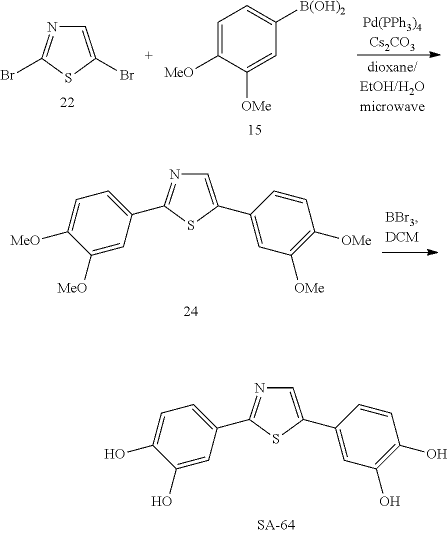

To a solution of dioxane/EtOH/H₂O (6 mL, 1/1/1) in a microwave reaction vial (Biotage) was added 2,5-dibromothiazole (22) (122 mg, 0.5 mmol), (3,4-dimethoxyphenyl)boronic acid (15) (218 mg, 1.2 mmol), Pd(PPh₃)₄ (29 mg, 0.025 mmol) and Cs₂CO₃ (0.72 g, 2.2 mmol). The mixture was purged with nitrogen and heated in a microwave reactor (Biotage) to 160° C. for 1 h. The reaction mixture was cooled to 23° C., acidified with 1M HCl until the pH was 1, and extracted with EtOAc. The combined organic extracts were washed with H₂O before being dried over MgSO₄, filtered, and concentrated under reduced pressure. Compound 24 was purified by column chromatography (5%-35% EtOAc in hexanes) as brown-yellow solid (28 mg, 16% yield). 5-bromo-2-(3,4-dimethoxyphenyl)thiazole (23) was also isolated from the reaction above as brown-yellow crystalline solid (28 mg, 19% yield).

SA-64 was prepared by the reaction of 2,5-bis(3,4-dimethoxyphenyl)thiazole (24) (28 mg, 0.078 mmol) and BBr₃ (1M in DCM, 0.75 mL, 0.75 mmol) according to the similar procedure for compound SA-58, and was obtained as a yellow solid (14 mg, 60% yield).

### Example 11

### Synthesis of SA-65

A mixture of 0.100 g (0.237 mmol) of 10, 0.600 g (1.03 mmol) of hexabutylditin, 5 mL of PhMe, and 1 mL of TEA was degassed by nitrogen purge. Then 0.050 g of Pd(PPh₃)₄ was added. The reaction mixture was refluxed for 16 h. At this time, the reaction was incomplete, but a decomposition product was seen in addition to the desired product. The reaction was at this point concentrated and purified by PTLC to prevent further decomposition to give 0.090 g (60% yield) of SA-65 as an off-white solid. ¹H NMR (200 MHz, CDCl₃) δ 7.68-7.65 (overlapping peak doublet and doublet of doublets, 2H), 7.53 (s, 1H), 7.16 (s, 1H), 6.98 (d, J=9.0 Hz, 1H), 3.99-3.96 (4 overlapping singlets, 12H), 1.55 (m, 6H), 1.44 (m, 6H), 1.15 (m, 6H), 0.84 (t, J=7.2 Hz, 9H).

### Example 9

### Synthesis of SA-66

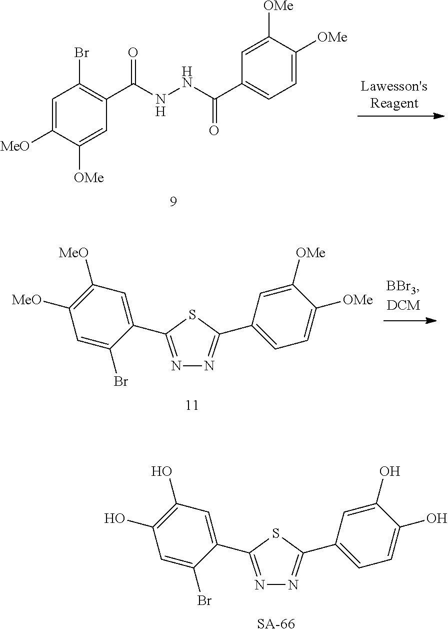

A mixture of 0.174 g of Lawesson's reagent, 0.175 g of compound 9, and 20 mL of PhMe was heated to 60° C. for 3 h. The mixture was concentrated, and applied directly to a PTLC plate for purification. The brominated thiadiazole 11 was purified by PTLC and the middle of the desired product spot (fluoresces blue under UV light) was collected to give 0.112 g (65% yield) of compound 11 as an off-white to brown solid.

The above experiment was repeated using 1.13 g of 9, 1.21 g of Lawesson's reagent, and 200 mL of PhMe. The mixture was refluxed 3 h, and quenched with 150 mL of water. The layers were separated, and the aqueous extracted twice with 30 mL of EtOAc. The organic layers were combined and washed twice with 50 mL 1 N aq. HCl, twice with 50 mL saturated aq. NaHCO₃, once with 25 mL of water, and once with 50 mL of brine. The organic was dried, concentrated, and purified by PTLC to give 1.04 g of the desired 11 as a yellow solid (97% yield). ¹H NMR (200 MHz, CDCl₃) δ 7.88 (s, 1H), 7.68 (bs, 1H), 7.51 (d, J=7.8 Hz, 1H), 7.15 (s, 1H), 6.95 (d, J=8.4 Hz, 1H), 4.00-3.95 (4 overlapping singlets, 12H).

A solution of 0.051 g of 11 in 5 mL DCM was cooled to −78° C., treated with 1.5 mL of 2 M BBr₃ in DCM, stirred at −78° C. for 0.5 h, and stirred at 23° C. for 3 h. The mixture was quenched with water carefully, poured into 100 mL brine, and extracted twice with 75 mL of EtOAc. The combined organic layers were dried and concentrated to yield 36 mg (81% yield) of the crude title compound as a yellow solid. The mixture was recrystallized from hot MeOH and precipitated with water to give 0.021 g (50% yield) of SA-66 as a brown solid.

The above experiment was repeated on 0.077 g of starting material using the same procedure with the exception of slightly different stirring times (1 h at −78° C., 4 h at 23° C.) to give 0.054 g (83% yield) of SA-66. ¹H NMR (200 MHz, DMSO-d₆) δ 10.10 (s, 1H), 9.78 (s, 1H), 9.68 (s, 1H), 9.47 (s, 1H), 7.55 (s, 1H), 7.43 (d, J=1.8 Hz, 1H), 7.29 (dd, J=1.8 Hz, 8.0 Hz, 1H), 7.13 (s, 1H), 6.87 (d, J=8.2 Hz, 1H).

### Example 10

### Synthesis of SA-67

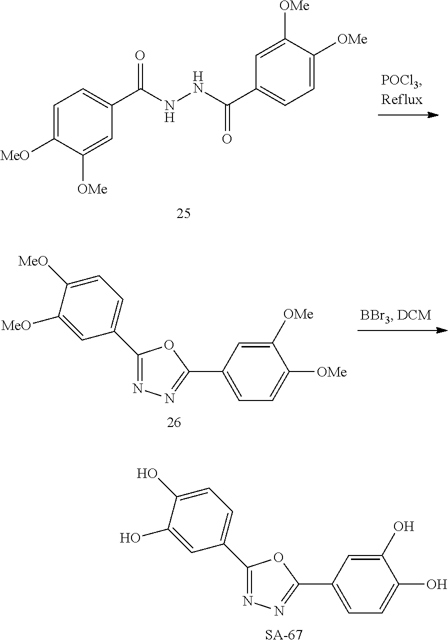

To 0.285 g (1.57 mmol) of 3,4 dimethoxybenzoic acid (1) in 5 mL of DCM was added 0.3 mL (3.5 mmol) of oxalyl chloride and one drop of DMF. The mixture was stirred for 2 h, quenched with hydrazine hydrate (3 mL), concentrated, and dried. The solid was taken up in 25 mL water, sonicated five minutes, filtered, and the resultant solid washed with 40 mL water. The solid was dried, taken up in 20:1 DCM:DMF, and purified by PTLC (8:2 EtOAc:Hexanes) to give 0.056 g (20% yield) of 25 as an off-white solid. ¹H NMR (200 MHz, DMSO-d₆) δ 10.30 (s, 2H), 7.58 (d, J=8.4 Hz, 1H), 7.51 (bs, 1H), 7.18 (d, J=8.4 Hz, 1H), 3.82 (bs, 12H).

To 0.050 g (0.14 mmol) of hydrazide 25 was added 3 mL of POCl₃. The mixture was refluxed 2 h, quenched with 50 g of ice, allowed to warm to 23° C., and extracted twice with 25 mL of EtOAc. The organic layers were washed once with 25 mL water, twice with 25 mL of brine, dried with Na₂SO₄, and concentrated to give 0.043 g (93% yield) of 26 as a white solid. ¹H NMR (200 MHz, CDCl₃) δ 7.72 (d, J=8.0 Hz, 2H), 7.60 (d, J=1.2 Hz, 1H), 7.07 (d, J=8.4 Hz, 1H), 3.89 (s, 6H), 3.87 (s, 6H).

To 0.038 g (0.11 mmol) of oxadiazole 26 was added 3 mL of DCM. The solution was cooled to −78°, and 5 mL of DCM containing 0.450 g (1.8 mmol) of BBr₃ was added. The mixture was stirred at −78° C. for 1 h, at 23° C. for 2.5 h, quenched by pouring into 5 mL of MeOH, and concentrated to 1 mL. The resultant oil was diluted with 5 mL water and the resultant precipitate was filtered to give 0.017 g (56% yield) of SA-67 as a yellow solid. ¹H NMR (200 MHz, DMSO-d₆) δ 9.74 (bs, 2H), 9.51 (bs, 2H), 7.43 (d, J=2.0 Hz, 1H), 7.37 (dd, J=2.0 Hz, 8.4 Hz, 1H), 6.93 (d, J=8.0 Hz, 1H).

### Example 11

### Synthesis of SA-68

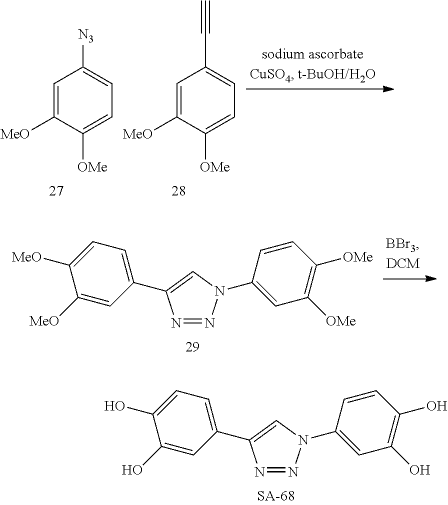

4-azido-1,2-dimethoxybenzene (27) (62 mg, 0.31 mmol), 4-ethynyl-1,2-dimethoxy benzene (28) (52 mg, 0.31 mmol), sodium ascorbate (0.25 mL of 1M solution, 0.25 mmol), CuSO₄ (0.020 mL of 1M solution, 0.020 mmol) and tBuOH/H₂O (2 mL, 1/1) were added to a vial. The reaction mixture was purged with nitrogen, stirred at 23° C. overnight, poured into water at 0° C., and the resultant brown solid was filtered. This solid was washed with water (1 mL) and Et₂O (1 mL). Compound 29 (99 mg, 93% yield) was used in the next step without further purification.

SA-68 was prepared by the reaction of 1,4-bis(3,4-dimethoxyphenyl)-1H-1,2,3-triazole (29) (67 mg, 0.20 mmol) and BBr₃ (1M in DCM, 0.79 mL, 0.79 mmol) according to the similar procedure for compound SA-52. SA-68 was obtained as a dark brown solid (56 mg, 98% yield).

### Example 12

### Synthesis of SA-69

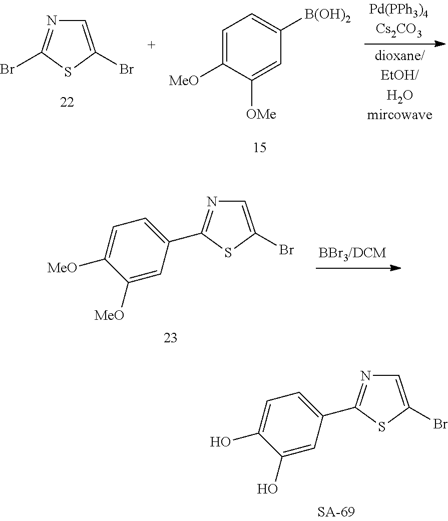

To a solution of dioxane/EtOH/H₂O (6 mL, 1/1/1) were added 2,5-dibromothiazole (22) (756 mg, 3.1 mmol), (3,4-dimethoxyphenyl)boronic acid (15) (380 mg, 2.1 mmol), Pd(PPh₃)₄ (58 mg, 0.05 mmol) and Cs₂CO₃ (1.4 g, 4.4 mmol) in a 20 mL microwave reaction vial (Biotage). The solution was purged with nitrogen and heated in a microwave reactor (Biotage) to 160° C. for 30 min. The reaction mixture was cooled to 23° C., acidified with 1M HCl until pH=1, and extracted with EtOAc. The combined organic extracts were washed with H₂O before being dried over MgSO₄, filtered, and concentrated under reduced pressure. Compound 23 was purified by flash column chromatography (8%-50% EtOAc in hexanes) as brown-yellow solid (193 mg, 32% yield). 2,5-bis(3,4-dimethoxyphenyl)thiazole (24), was also isolated as a brown-yellow solid (34 mg, 5% yield).

SA-69 was prepared by the reaction of 5-bromo-2-(3′,4′-dimethoxyphenyl)thiazole (23) (78 mg, 0.26 mmol) and BBr₃ (1M in DCM, 0.65 mL, 0.65 mmol) according to the similar procedure for compound SA-52, and was obtained as a brown-yellow solid quantitatively.

### Example 13

### Synthesis of SA-70

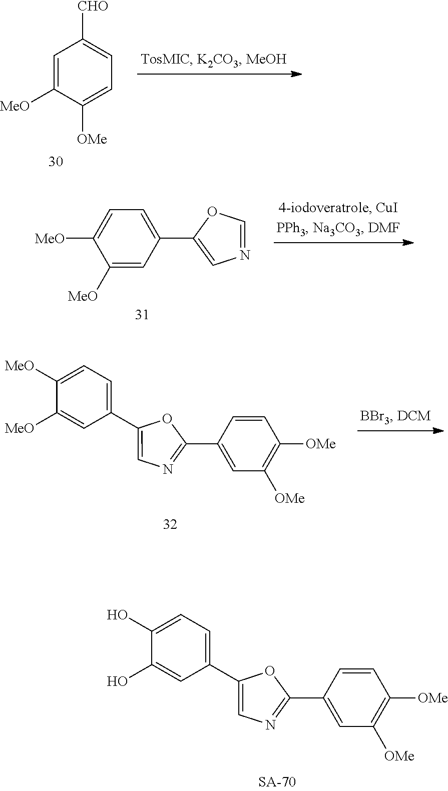

A mixture of 0.996 g (6 mmol) 3,4-dimethoxybenzaldehyde (30), 1.25 g of TosMIC (6.4 mmol), and 0.923 g (6.6 mmol) of K₂CO₃ was refluxed in 30 mL of MeOH for 3 h. The reaction mixture was quenched by pouring into 200 mL of a 1:1 mixture of brine and water, cooled to −20° C. for 1 h, and the resultant solid filtered to give 0.952 g (77% yield) of 31 as an off-white solid. ¹H NMR (200 MHz, CDCl₃) δ 7.85 (s, 1H), 7.22 (s, 1H), 7.20 (overlapping peak, 1H), 7.15 (dd, J=2.0 Hz, 12.4 Hz, 1H), 6.88 (d, J=1H), 3.91 (s, 3H), 3.88 (s, 3H).

A suspension of 0.212 g of Na₂CO₃ (2 mmol), 0.262 g of PPh₃ (1 mmol), 0.206 g (1 mmol) of 31 and 0.316 g of 4-iodoveratrole (1.2 mmol) was formed in 1 mL of DMF. Then, 0.190 g (1 mmol) of CuI was added. The mixture was stirred at 160° C. for 3 h and quenched by pouring into 50 mL of water containing 5% NH₄⁺OH⁻. The product was extracted with 50 mL DCM, the organic layer dried and concentrated, and the crude product purified by PTLC to give 0.174 g (51% yield) of 32 as a yellow solid. ¹H NMR (200 MHz, CDCl₃) δ 7.60 (d, J=8.4 Hz, 1H), 7.54, (bs, 1H), 7.24 (s, 1H), 7.19 (bs, 1H), 7.10 (s, 1H), 6.90-6.83 (m, 2H), 3.91-3.86 (4 overlapping multiplets, 12H). ¹³C NMR (50 MHz, CDCl₃) δ 160.7, 150.9, 149.3, 149.2, 132.1, 132.0, 122.0, 121.2, 120.4, 119.3, 117.1, 111.5, 111.1, 109.1, 107.5, 56.01, 55.92.

A mixture of 0.075 g (0.22 mmol) of 32 in 15 mL DCM was treated with 0.500 g (2 mmol) of BBr₃ at −78° C. The mixture was stirred at −78° C. for 0.5 h, then 2 h at 23° C. The reaction was then quenched with 5 mL MeOH, concentrated to 1 mmol and diluted with 5 mL of water. The resultant precipitate was filtered to give 0.021 g (33% yield) of SA-70 as a yellow solid. ¹H NMR (200 MHz, DMSO-d₆) δ 7.40 (s, 2H), 7.31 (dd, J=2.0 Hz, 8.0 Hz, 1H), 7.13 (d, J=2.2 Hz), 7.05 (dd, J=2.2 Hz, 8.2 Hz, 1H), 6.86 (d, J=7.8 Hz, 1H), 6.82 (d, J=7.8 Hz, 1H). HRMS Calculated for C₁₅H₁₂NO₅ (M+H)⁺ 286.0715. found 286.0717.

### Example 14

### Synthesis of SA-72

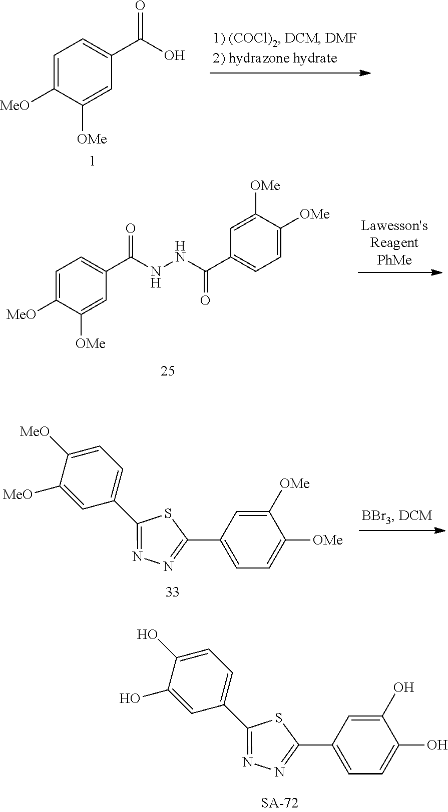

A solution of 0.115 g (0.32 mmol) of 25 in 30 mL of PhMe was treated with 0.140 g of Lawesson's reagent (0.35 mmol) and stirred 3 h at 100° C. The reaction mixture was poured into 75 mL water, shaken vigorously, and extracted with 50 mL EtOAc. The organic layers were combined, washed with 25 ml saturated aqueous NaHCO₃, washed with 50 mL brine, dried, and concentrated. The crude product was purified by PTLC to give 0.088 g (74% yield) of 33 as a yellow solid. ¹H NMR (200 MHz, CDCl₃) δ 7.60 (bs, 2H), 7.37 (d, J=8.2 Hz, 2H), 6.86 (d, J=8.4 Hz, 2H), 3.95 (s, 6H), 3.90 (s, 6H). ¹³C NMR (200 MHz, CDCl₃) 167.3, 151.6, 149.4, 123.2, 121.6, 111.2, 110.0, 56.1, 56.0. HRMS Calculated for C₁₆H₁₉N₂O₄S (M+H)⁺ 359.1066. found 359.1061.

A solution of 0.077 g (0.22 mmol) of 33 and 3 mL DCM was treated with 0.250 g BBr₃ at −78° C. The reaction mixture was stirred 1 h, treated with 0.250 g BBr₃, and stirred an addition 15 min at −78° C. The mixture was warmed to 23° C., stirred for 1 h, quenched with 10 mL MeOH, and concentrated. The concentrate was taken up in 1 mL MeOH, warmed until a solution, and precipitated with 10 mL water. The resultant solid was filtered and dried to give 0.054 g of SA-72 (83% yield) as an off-white solid. ¹H NMR (200 MHz, DMSO-d₆) 10.0-9.0 (broad peak, 4H), 7.41 (d, J=2.2 Hz, 2H), 7.24 (dd, J=2.2 Hz, 8.2 Hz, 1H), 6.87 (d, J=8.2 Hz, 2H).

### Example 15

### Synthesis of SA-74

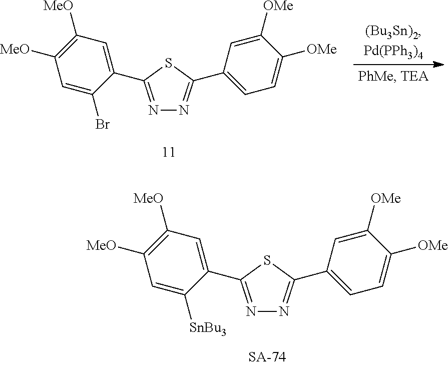

To a mixture of 0.219 g (0.5 mmol) of 11 in 20 mL PhMe was added 1.09 g (Bu₃Sn)₂ and 2 mL of triethylamine. The mixture was degassed via a nitrogen purge, treated with 0.150 g (0.13 mmol) of Pd(PPh₃)₄, and refluxed for 15 h. The mixture was concentrated and purified directly by PTLC by eluting the plates with 1% TEA in hexanes then 50% EtOAc in hexanes with 1% TEA successively. This gave 0.224 g (69% yield) SA-74 as an off-white solid.

### Example 16

### Synthesis of SA-75 and SA-76

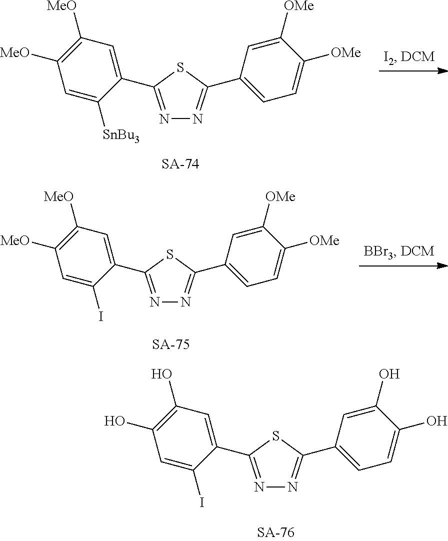

A solution of 0.051 g of SA-74 in 3 mL of DCM was treated with a 1 M iodine in DCM solution until the orange/yellow color of the iodine persists. The mixture was quenched with a 1 M solution of KF in MeOH (2 mL) then sat. aq. Na₂S₂O₃ (2 mL). The mixture was extracted twice with 10 mL EtOAc. The organic layers were combined, washed with 10 mL water, dried and concentrated to give the crude title compound. The crude product was purified by PTLC with another run of this reaction using 0.082 g of SA-74 and run in the same manner as described above. This gave 0.012 g (12% yield) of SA-75 as a yellow solid. ¹H NMR (200 MHz, CDCl₃) δ 7.73 (d, J=2.2 Hz, 1H), 7.59 (s, 1H), 7.54 (dd, J=2.2 Hz, 8.4 Hz), 7.42 (s, 1H), 6.98 (d, J=8.4 Hz, 1H), 4.03-3.95 (4 overlapping singlets, 12H).

A solution of 0.008 g of SA-75 in 1 mL of DCM was cooled to 0° C., treated with 0.5 mL of 1 M BBr₃ in DCM, and stirred for 1 h. The reaction was treated with an additional 1 mL portion of 1 M BBr₃ in DCM, stirred an additional 2 h at 0° C., and warmed to 23° C. The reaction mixture was stirred an additional 0.5 h, quenched with 3 mL MeOH, and concentrated to 0.5 mL. The product was precipitated with 5 mL water, and the resultant yellow solid was filtered and dried to give (0.002 g) of SA-76 as a yellow-green solid. ¹H NMR (200 MHz, DMSO-d₆) δ 9.93-9.50 (overlapping broad singlets, 4H), 7.42-7.28 (overlapping irresolvable peaks, 4H), 6.98 (d, J=8.2 Hz, 1H).

### Example 17

### Synthesis of SA-77 and SA-78

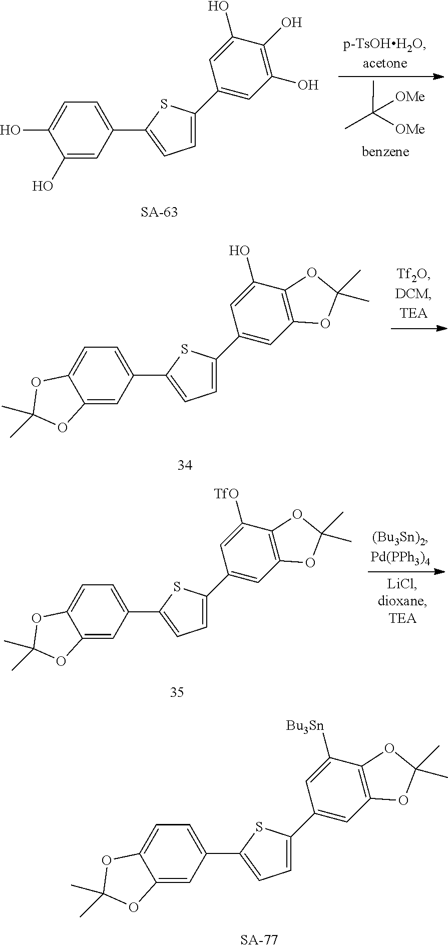

To a round-bottom flask charged with 5-(5-(3,4-dihydroxyphenyl)thiophen-2-yl)benzene-1,2,3-triol (SA-63) (145 mg, 0.46 mmol), 4-methylbenzenesulfonic acid (p-TsOH.H₂O) (8.7 mg, 0.046 mmol), and 2,2-dimethoxypropane (0.45 mL, 3.7 mmol) were added acetone (3 mL) and benzene (15 mL). A short column with 4 Å molecular sieves and a condenser were installed on the flask. The reaction was refluxed for 15 h. An additional portion of 2,2-dimethoxypropane (0.45 mL, 3.7 mmol) was added, and the reaction was refluxed for another 24 h. The solution was concentrated at reduced pressure and purified by PTLC, which afforded 34 as a white solid (53 mg, 29%).

To a solution of 6-(5-(2,2-dimethylbenzo[d][1,3]dioxol-5-yl)thiophen-2-yl)-2,2-dimethylbenzo[d][1,3]dioxol-4-ol (34) (53 mg, 0.13 mmol), triethylamine (0.056 mL, 0.40 mmol) and DCM (2 mL) was added trifluoromethanesulfonic anhydride (0.034 mL, 0.20 mmol) at 0° C. The reaction was warmed to 23° C. after 30 min and extracted with DCM. The organic layer was washed with sat. aq. NaHCO₃, H₂O and brine. The organic layers were dried over MgSO₄, concentrated, and 35 was obtained as yellowish oil (53 mg, 75% yield).

To 6-(5-(2,2-dimethylbenzo[d][1,3]dioxol-5-yl)thiophen-2-yl)-2,2-dimethylbenzo[d][1,3]dioxol-4-yl trifluoromethanesulfonate (35) (32 mg, 0.061 mmol) was added Pd(PPh₃)₄ (14 mg, 0.012 mmol), LiCl (26 mg, 0.61 mmol), bistributylditin (0.153 mL, 0.305 mmol) and dioxane (2 mL) (plus Triethylhylamine??). The solution was purged with nitrogen and refluxed for 3.5 h. The reaction was concentrated, evaporated and subjected to PTLC (8% EtOAc in hexanes). SA-77 was obtained as a yellowish oil (26 mg, 64% yield).

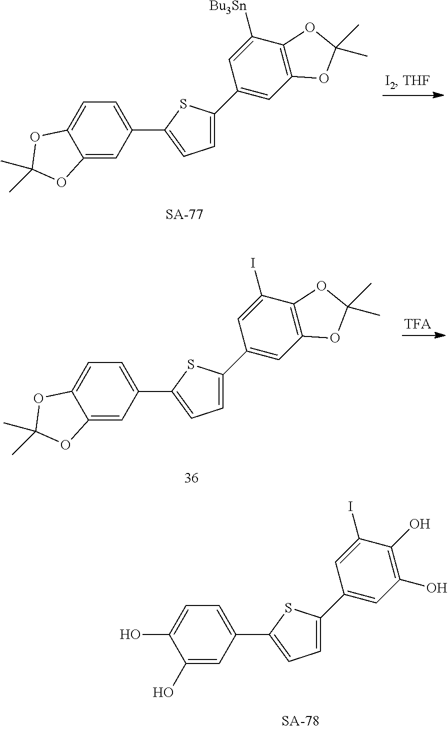

To the solution of tributyl(6-(5-(2,2-dimethylbenzo[d][1,3]dioxol-5-yl)thiophen-2-yl)-2,2-dimethylbenzo[d][1,3]dioxol-4-yl)stannane (SA-77) (26 mg, 0.039 mmol) and THF (1 mL) was added the solution of I₂ (20 mg, 0.078 mmol) in THF (1 mL) dropwise. The reaction was stirred for 10 min and concentrated, and 36 was obtained quantitatively as a yellow solid after purification by PTLC (7% EtOAc in hexanes).

To 6-(5-(2,2-dimethylbenzo[d][1,3]dioxol-5-yl)thiophen-2-yl)-4-iodo-2,2-dimethylbenzo [d][1,3]dioxole (36) (15 mg, 0.029 mmol) were added a few drops of H₂O and trifluoroacetic acid (0.45 mL). The reaction was stirred at 23° C. for 1 h, and afforded SA-78 quantitatively as a white crystal after concentration at reduced pressure.

### Example 18

### Synthesis of SA-79 and 80

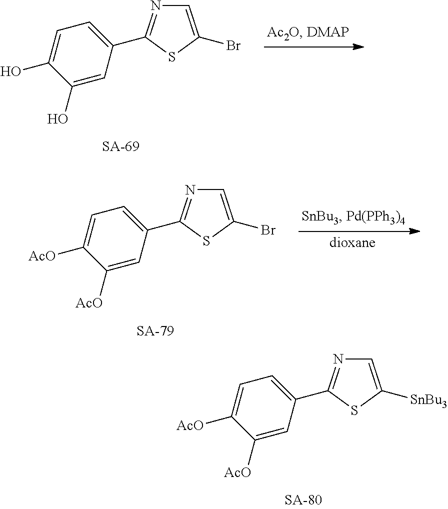

To 4-(5-bromothiazol-2-yl)benzene-1,2-diol (SA-69) (90 mg, 0.32 mmol) was added 4-dimethylaminopyridine (DMAP) (117 mg) and acetic anhydride (3 mL). The reaction was stirred at 23° C. for 1 h before it was extracted with EtOAc. The organic layer was washed with sat. aq. NaHCO₃, H₂O and brine and dried over MgSO₄. SA-79 was obtained as yellow solid (74 mg, 65% yield) after PTLC (20% EtOAc in hexanes).

To a solution of 4-(5-bromothiazol-2-yl)-1,2-phenylene diacetate (SA-79) (36 mg, 0.10 mmol), Pd(PPh₃)₄ and dioxane (2 mL) was added bistributyltin (0.25 mL, 0.50 mmol). The solution was purged with nitrogen and refluxed for 1.5 h. PTLC (25% EtOAc in hexanes) provided SA-80 as yellow oil (22 mg, 39% yield).

### Example 19

### Synthesis of SA-81

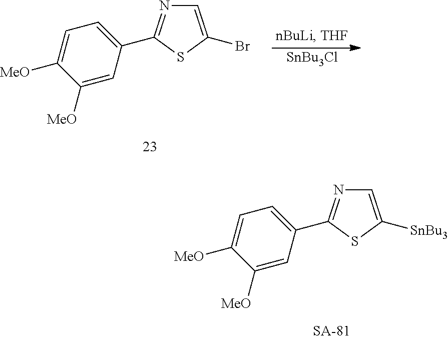

To a solution of nBuLi (0.34 mL, 2.5 M in hexanes) and THF (6 mL) was added 5-bromo-2-(3,4-dimethoxyphenyl)thiazole (23) (54 mg, 0.18 mmol) and additional THF (3 mL) was added dropwise at −78° C. under nitrogen atmosphere. SnBu₃Cl (0.15 mL) was added after the reaction was stirred at −78° C. for 30 min. After another 30 min, saturated aqueous NaHCO₃ was added to quench the reaction. The mixture was extracted with EtOAc and the organic layer was washed with water and brine. PTLC (25% EtOAc in hexanes) provided SA-81 as a yellow oil (58 mg, 63% yield).

### Example 20

### Synthesis of SA-82

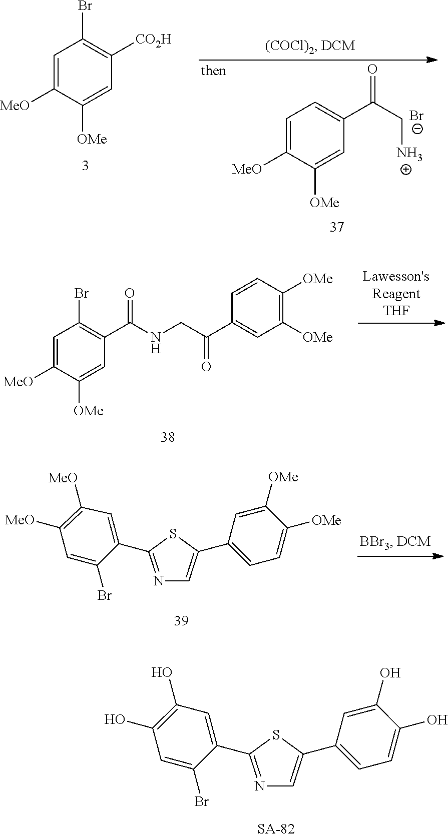

A mixture of 0.26 g (1 mmol) of 2-bromo-4,5-dimethoxybenzoic acid (3) in 5 mL of DCM was treated with 0.3 mL of (COCl)₂ and two drops of DMF. The mixture was stirred for an additional hour after a yellow solution had formed (about 2 h total). The mixture was concentrated and dried in vacuo thoroughly, taken up in 15 mL of DCM, cooled to 0° C., and treated with 2 mL pyridine. Then 0.79 g of known amine 37 was added. The mixture was warmed to 23° C., stirred 3 days, and quenched with water. The product was extracted with 10 mL of DCM, the combined organic was washed with 10 mL water, dried, and concentrated to give the crude product that was purified by PTLC (50% EtOAc in hexanes) to give 0.070 g (16% yield) of compound 38 as a light brown solid. ¹H NMR (200 MHz, CDCl₃) δ 7.67 (dd, J=2.0 Hz, 8.4 Hz, 1H), 7.56 (apparent broad singlet, overlapping peaks, 2H), 7.04 (s, 1H), 6.95 (d, J=6.95 Hz, 1H), 4.94 (d, J=4.2 Hz, 1H), 3.98 (s, 3H), 3.95 (s, 3H), 3.86 (bs, 6H). HRMS Calculated for C₁₉H₂₂O₆NBr (M+H)⁺ 438.0552. found 438.0556.

A mixture of 0.103 g (0.24 mmol) of amide 38 in 10 mL of THF was treated with 0.117 g (0.29 mmol) of Lawesson's reagent. The mixture was refluxed for 2 h, cooled, and concentrated. The crude product was purified directly by PLTC to give 0.048 g (45% yield) of compound 39 as a light brown solid. ¹H NMR (200 MHz, CDCl₃) δ 7.78 (apparent singlet, 1H), 7.55-7.50 (overlapping singlet, dd, 2H), 7.46 (s, 1H), 7.13 (s, 1H), 6.94 (d, J=8.8 Hz, 1 h), 3.97-3.89 (overlapping singlets, 12H).

A solution of 0.031 g 39 (0.07 mmol) in 4 mL of DCM was cooled to −78° C., and treated with 1.5 mL of 1 M BBr₃ in DCM. The mixture was stirred for 1 h at −78° C., 1 h at 23° C., and quenched with 2 mL of MeOH. The mixture was concentrated to 1 mL, diluted with 10 mL water, sonicated, and the resultant solid filtered to give 0.009 g (35% yield) of SA-82 as an off-white solid.

### Example 21

### Synthesis of SA-83

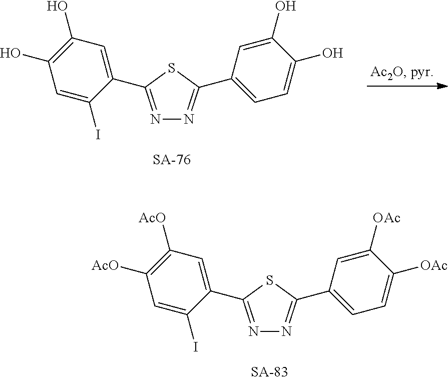

To 0.012 g (0.03 mmol) of SA-76 in 0.5 mL pyridine was added 0.05 mL of acetic anhydride. The mixture was stirred at 140° C. for 3 h, cooled, and poured into 10 mL of water with 0.5 g NH₄⁺Cl⁻, and the product was extracted with 5 mL 10% MeOH in DCM. The extract was concentrated, and purified by PTLC to give 0.005 g (8.3*10⁻³ mmol) of SA-83 as a brown oil. ¹H NMR (200 MHz, CDCl₃) δ 7.94-7.88 (3 overlapping irresolvable peaks, 3H), 7.82 (s, 1H), 7.36 (d, J=9.0 Hz), 2.34-2.32 (4 overlapping singlets, 12H).

### Example 21

### Synthesis of SA-84

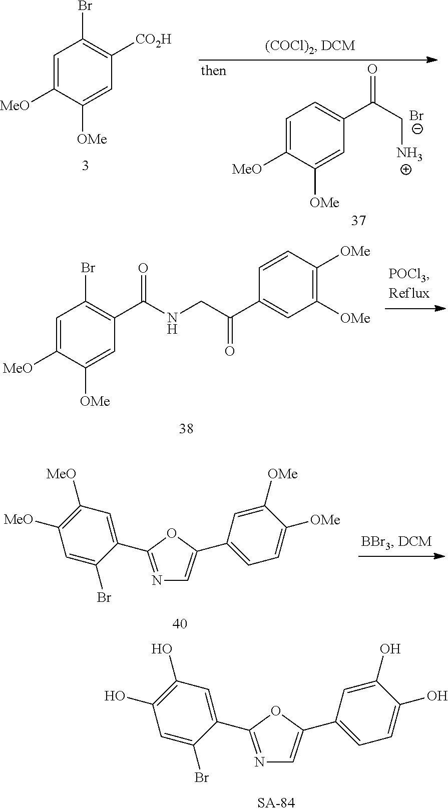

A mixture of 0.058 g (0.132 mmol) of amide 38 was refluxed in 2 mL POCl₃. The solution was added to 50 mL of water and sonicated to precipitate a light yellow solid, which was filtered, washed with cold water, and dried to give 0.041 g (74% yield) of 40 as a yellow solid. ¹H NMR (200 MHz, CDCl₃) δ 7.61 (s, 1H), 7.37 (d, J=2.0 Hz, 1H), 7.31 (dd, J=2.2 Hz, 8.4 Hz, 1H), 7.25 (s, 1H), 7.18 (s, 1H), 6.96 (d, J=8.4 Hz, 1H), 3.96 (overlapping singlets, 12H). HRMS Calculated for C₁₉H₁₉BrNO₅ (M+H)⁺ 420.0448. found 420.0465.

To 0.100 g (0.24 mmol) of 40 in 20 mL of DCM at −78° C. was added 2.5 mL of 1 M BBr₃ in DCM. The reaction was warmed to 23° C., stirred for 3 h, and quenched with 10 mL of MeOH. The reaction mixture was concentrated, and water was added to precipitate the product. This did not yield any solid, so the crude product was reconcentrated and purified by PTLC (10% MeOH in DCM) to give, after 2 weeks drying, 0.040 g (46% yield) of SA-84 as a yellow-green solid. ¹H NMR (200 MHz, DMSO-d₆) δ 7.50 (s, 1H), 7.44 (s, 1H), 7.16 (d, J=2.0 Hz), 7.09 (s, 1H), 7.08 (dd, coupling constants not resolvable due to overlapping singlet, 1H), 6.83 (d, J=8.2 Hz, 1H).

### Example 22

### Synthesis of SA-86

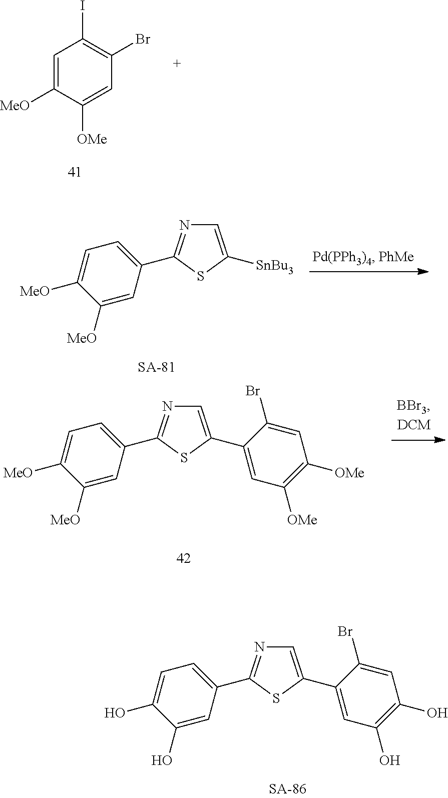

To a mixture of 1-bromo-2-iodo-4,5-dimethoxybenzene (41) (27 mg, 0.078 mmol), Pd(PPh₃)₄ (4.1 mg, 5 mol %) and toluene (1 mL) was added the solution of 2-(3,4-dimethoxyphenyl)-5-(tributylstannyl)thiazole (SA-81) (36 mg, 0.070 mmol) and toluene (1 mL). The reaction was purged with nitrogen and refluxed 16 h. The concentrated reaction mixture was purified by PTLC (35% EtOAc in hexanes) and afforded 42 (20 mg, 65% yield).

SA-86 was prepared by the reaction of 5-(2-bromo-4,5-dimethoxyphenyl)-2-(3,4-dimethoxyphenyl) thiazole (42) (20 mg, 0.046 mmol) and BBr₃ (in DDM??) (0.46 mL, 0.46 mmol) according to the similar procedure for compound SA-58, and obtained SA-86 as a yellow solid quantitatively.

### Example 23

### Synthesis of SA-99

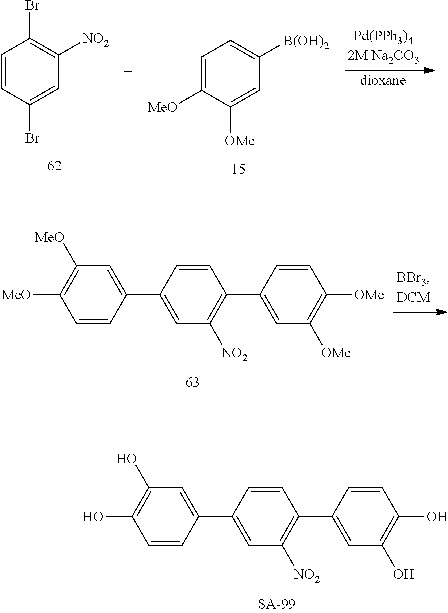

A mixture of dibromide 62 (0.562 g, 2 mmol), boronic acid 15 (0.910 g, 5 mmol), 20 mL of dioxane, and 24 mL of 2 M Na₂CO₃ (aqueous) was degassed by nitrogen purge. Then Pd(PPh₃)₄ (0.12 g, 0.10 mmol) was added. The resultant mixture was refluxed 20 h, diluted with 50 mL of water, and extracted three times with 50 mL of ethyl acetate. The combined organic layers were washed once with 50 mL of water, dried, and concentrated to give the crude product. This was purified by Flash 40+M chromatography (Biotage) using gradient ethyl acetate in hexanes as the eluent to give 63 as a yellow solid (0.288 g, 36% yield).

¹H NMR (CDCl₃, δ), 7.94 (d, J=1.8 Hz, 1H), 7.75 (dd, J=1.8, 8.0 Hz, 1H), 7.47 (d, J=8.0 Hz, 1H), 7.17 (dd, J=2.2, 8.2 Hz, 1H), 7.10 (d, J=2.0 Hz, 1H), 6.96 (d, J=8.0 Hz, 1H), 6.90-6.85 (overlapping peaks, 3H), 3.95 (s, 3H), 3.92 (s, 3H), 3.90 (s, 3H), 3.87 (s, 3H).

¹³C NMR (CDCl₃, δ) 149.9, 149.7, 149.6, 149.3, 149.1, 141.1, 133.9, 132.2, 131.2, 130.0, 129.5, 121.8, 120.5, 119.6, 111.7, 111.4, 111.2, 110.1, 56.11, 56.05, 56.0, 55.9.

A solution of 63 (0.062 g, 0.16 mmol) in 25 mL DCM was cooled to −78° C., treated with BBr₃ (1.125 g, 4.5 mmol) in 6 mL of DCM, stirred 2.5 h at −78° C., warmed to 23° C., stirred at −23° C. for 1.5 h, quenched with 20 mL water, and extracted once with 100 mL of ethyl acetate. The ethyl acetate layer was dried and concentrated to 3 mL and precipitated with excess DCM. The precipitate was filtered and dried to give SA-99 as a yellow solid (0.037 g, 70% yield).

¹H NMR (DMSO-d₆) 9.17-9.11 (bs, 4H), 7.96 (d, J=1.6 Hz, 1H), 7.83 (dd, J=2.2, 8.2 Hz, 1H), 7.49 (d, J=8.0 Hz, 1H), 7.14 (bs, 1H), 7.08 (d, J=8.4 Hz, 1H), 6.88-6.79 (overlapping peaks, 2H), 6.72 (d, J=2.2 Hz, 1H), 6.63 (dd, J=2.0, 8.2 Hz, 1H),

HRMS Calculated for C₁₈H₁₃NO₆Na (M+Na)⁺ 362.0641. Found 362.0645.

### Example 24

### Synthesis of SA-108

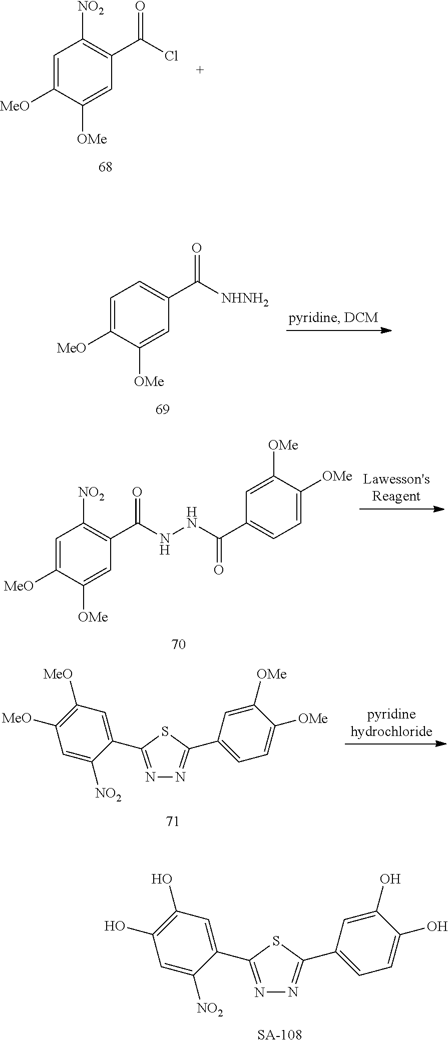

To 1.00 g (4.07 mmol) of acid chloride 68 in 25 mL of DCM at 0° C. was added 5 mL of pyridine. The mixture was stirred 3 min, and 0.76 g (3.87 mmol) of known hydrazide 69 was added at once. The mixture was gradually allowed to warm to 21-23° C. and stirred at this temperature for 16 h. The mixture was concentrated, treated with 10 mL of ethanol, warmed to reflux, and diluted with 30 mL of water. After returning the mixture to reflux, ethanol was added gradually until a solution formed. The solution was allowed to cool, and the precipitated light yellow solid was collected and dried to give 0.498 g (1.24 mmol, 32% yield) of 70.

¹H NMR (CDCl₃ and MeOD, 6) 7.54 (s, 1H), 7.45 (dd, J=2.2, 8.4 Hz, 1H), 7.39 (d, J=1.8 Hz), 7.16 (s, 1H), 6.82 (d, J=8.4 Hz, 1H), 3.98-3.73 (overlapping singlets+residual H₂O peak, 12H).

A mixture of 0.398 g (1 mmol) of 70 and 0.452 g (1.11 mmol) of Lawesson's reagent in 50 mL of THF were refluxed for 16 h. The mixture was concentrated and purified by PTLC using 10% EtOAc in DCM as the eluent to give 0.255 g (0.62 mmol, 62% yield) 71 as a bright yellow solid.

¹H NMR (DMSO-d₆) 7.77 (s, 1H), 7.60-7.58 (overlapping peaks, 2H), 7.40 (s, 1H), 7.15 (d, J=8.8 Hz, 1H), 3.97 (s, 3H), 3.95 (s, 3H), 3.89 (s, 3H), 3.87 (s, 3H).

A mixture of 0.100 g (0.24 mmol) of 71 in 5 g of pyridine hydrochloride was heated to 200° C. for 30 min. The mixture was cooled, treated with 30 mL of water, and filtered. The resultant solid was recrystallized from aqueous methanol to yield 0.016 g of a brown solid as SA-108. The product is only sparingly soluble in DMSO-d₆, and insoluable in other deuterated solvents or solvent mixtures.

¹H NMR (DMSO-d₆, 6), 8.79 (s, 1H), 7.80 (bs, 1H), 7.57 (s, 1H), 7.43 (m, 1H), 7.28 (m, 1H), 6.91 (s, 1H), 6.80 (d, J=8.0 Hz, 1H). Example 1-Compounds provided herein bind are potent disruptors/inhibitors of Parkinson's disease α-synuclein fibrils

### Example 25

### Compounds Provided Herein Bind with High Affinity to Parkinson's Disease α-Synuclein Fibrils

The compounds prepared in the preceding examples were found to bind with high affinity to α-synuclein aggregates/fibrils that are found in the hallmark Lewy Bodies of Parkinson's disease. In order to assess relative binding affinities of the test compounds for aggregated α-synuclein, competition assays were set up with a radiolabeled molecule already known to bind to α-synuclein fibrils and non-radiolabeled test compounds. In order to induce its aggregation, α-synuclein was incubated in phosphate buffered saline (PBS, pH 7.4) at 37° C. for three days with shaking (1,400 rpm). Competitive binding assays were carried out in 12×75 mm borosilicate glass tubes. The reaction mixture contained 100 μL of α-synuclein aggregates (0.5-1 μg), [³H] positive reference compound #1 (100-200 nM diluted in PBS) and 50 μL of competing compounds (10⁻⁵-10⁻⁹ M, diluted serially in PBS containing 0.1% bovine serum albumin) in a final volume of 0.25 ml. Non-specific binding was defined in the presence of cold positive reference compound #1 (50 μM) in the same assay tubes. The mixture was incubated for 120 min at 37° C., and the bound and the free radioactivity were separated by vacuum filtration through Whatman GF/B filters using a Brandel M-24R cell harvester, followed by washing with PBS buffer three times. Filters containing the bound [³H] positive reference compound #1 were assayed for radioactivity in a liquid scintillation counter (Beckman LS6500). IC₅₀ values were determined by a non-linear, least squares regression analysis. Inhibition constants (Ki) values were calculated using the equation of Cheng and Prusoff (Cheng et al., Biochemical Pharmacology 22:3099-3108, 1973) using the observed IC₅₀ of the tested compound, the concentration of radioligand employed in the assay, and the value for the Kd of the ligand (600 nM).

The results from these experiments are reported in Table 1. As an example, SA-64 binds with high affinity to α-synuclein fibrils (Ki=89 nM) but does not show significant binding affinity for the amyloid-β peptide of Alzheimer's disease (not shown) which makes this compound more useful as a tool to aid in the specific detection of Parkinson's disease. Similarly, SA-58 and SA-57 bind with high affinity to α-synuclein aggregates, with binding constants (Ki) of 105 nM and 124 nM, respectively. The increased binding affinity (by 4-5-fold) of SA-57 and SA-58, relative to positive reference molecule #1 represents a significant improvement in binding to α-synuclein aggregates for these new molecules. Taken together, these results indicate that SA compounds bind to varying degrees to the α-synuclein aggregates, a hallmark of synucleinopathies such as Parkinson's disease.

TABLE 1
SA compounds bind to α-synuclein aggregates as measured
by an in vitro competition binding assay.

| SA # | Kᵢ (nM) |
|---|---|
| 52 | 1260 |
| 53 | 813 |
| 54 | 1640 |
| 55 | 370 |
| 57 | 124 |
| 58 | 105 |
| 59 | 697 |
| 61 | 3100 |
| 62 | 506 |
| 63 | 283 |
| 64 | 89 |
| 66 | 135 |
| 67 | 466 |
| 68 | 697 |
| 69 | 765 |
| 70 | 659 |
| 72 | 270 |
| 76 | 219 |
| 78 | 330 |
| 79 | 537 |
| 82 | 177 |
| 83 | 330 |
| 84 | 639 |
| 86 | 224 |
| 87 | 2000 |
| 88 | 970 |
| 89 | 1200 |
| 90 | 1700 |
| 94 | 1100 |
| 95 | 890 |
| 96 | 1350 |
| Positive reference #1 | 532 |
| Negative reference #1 | no binding |
| Negative reference #2 | >10000 |

The claimed subject matter is not limited in scope by the specific embodiments described herein. Indeed, various modifications of the specific embodiments in addition to those described will become apparent to those skilled in the art from the foregoing descriptions. Such modifications are intended to fall within the scope of the appended claims. Various publications are cited herein, the disclosures of which are incorporated by reference in their entireties.

1. A method of in vivo imaging of α-synuclein aggregates, comprising introducing into a mammal a detectable amount of a compound selected from 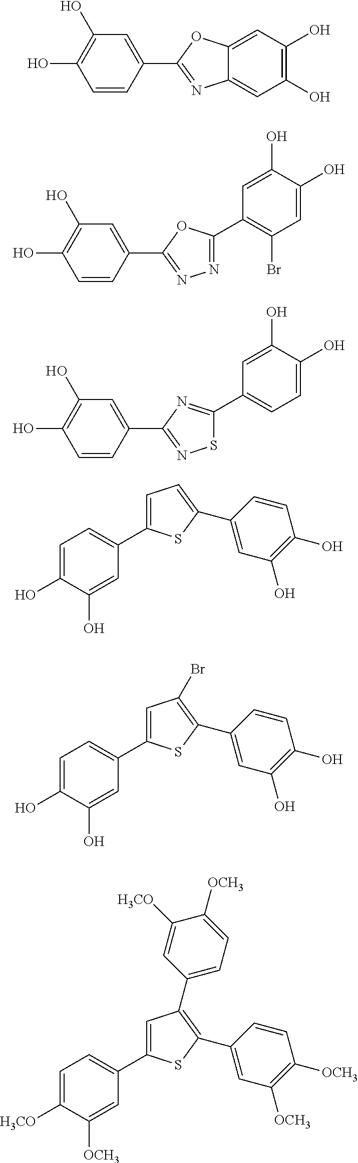 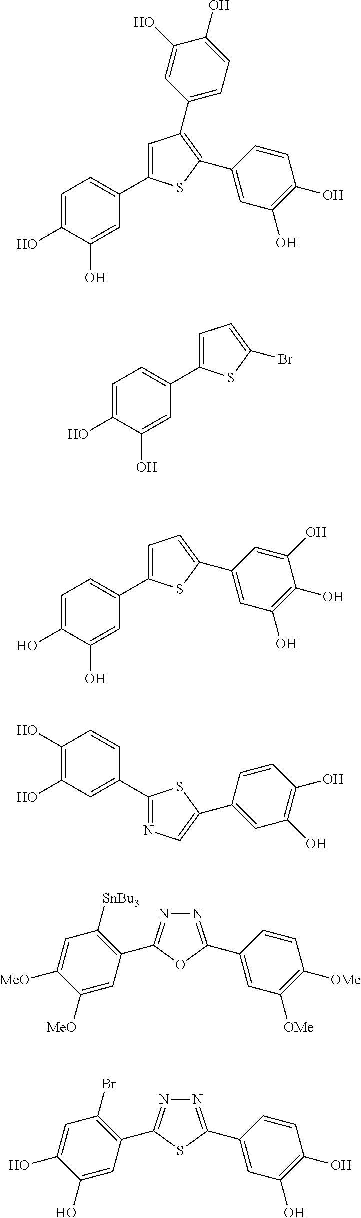 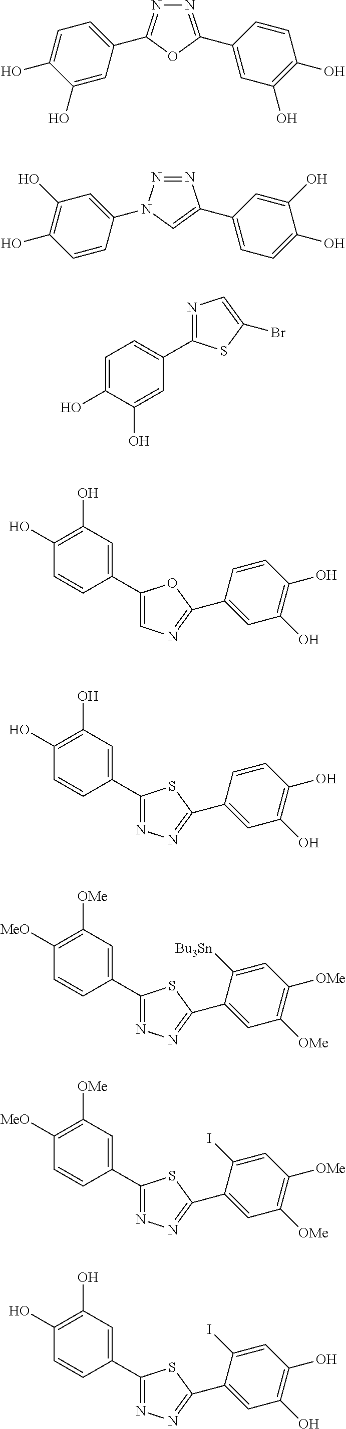 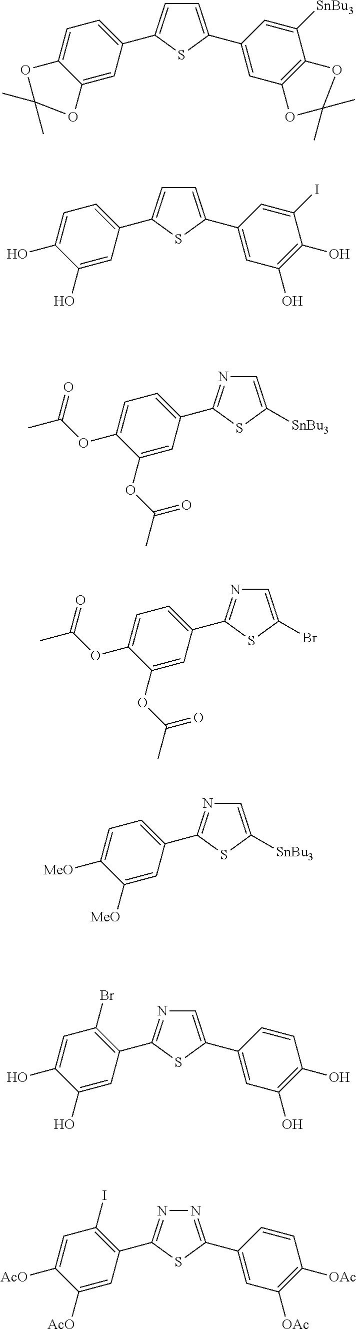 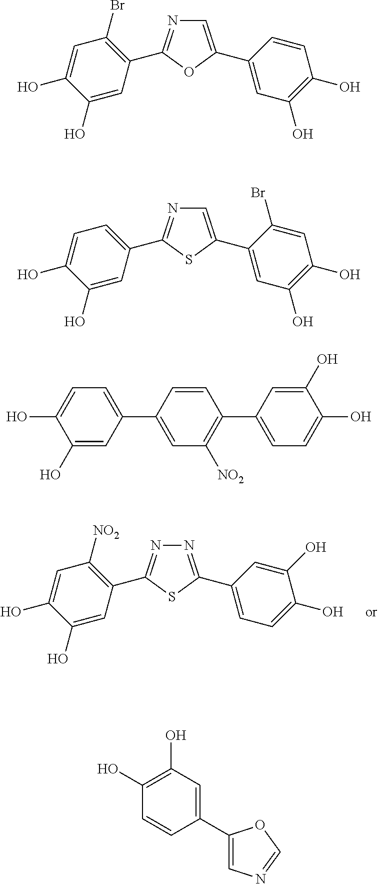
which has been labeled with a detectable substance, allowing a sufficient time period for the labeled compound to become associated with α-synuclein aggregates, and detecting the labeled compound associated with one or more α-synuclein aggregates.

2. The method of claim 1, comprising the additional step of quantifying the amount of labeled compound associated with the one or more α-synuclein aggregates.

3. The method of claim 1, wherein the step of detecting the labeled compound is by positron emission tomography or single photon emission computed tomography.

4. The method of claim 1, wherein the detectable substance is selected from the group consisting of: radioisotopes, phosphorescent compounds, fluorescent compounds, fluorescent proteins, paramagnetic compounds, metal chelators, and enzymes.

5. The method of claim 4, wherein the radioisotope is selected from the group consisting of: isotopes of Br, ¹²³I, ¹²⁵I, ¹³¹I, ^{99m}Tc, ¹¹C and ¹⁸F.

6. The method of claim 1, wherein the sufficient time period for the labeled compound to become associated with α-synuclein aggregates is from about 5 minutes to a time corresponding to approximately twice the radioactive half-life of the radioisotope of the labeled compound.

7. The method of claim 1, wherein the sufficient time period for the labeled compound to become associated with α-synuclein aggregates is from about 10 minutes to a time corresponding to the radioactive half-life of the radioisotope of the labeled compound.

8. The method of claim 1, wherein the sufficient time period for the labeled compound to become associated with α-synuclein aggregates is about 1 to about 60 minutes.

9. The method of claim 1, wherein the detectable amount of labeled compound comprises from about 0.1 to about 20 mCi.

10. The method of claim 1, wherein the labeled compound is in dosage form for intravenous injection.

11. A kit for detecting α-synuclein aggregates in the brain of an individual, comprising: a labeled compound; and instructions for using the labeled compound, said instructions comprising a direction to administer to an individual a detectable amount of labeled compound; a direction to wait a sufficient period of time; and a direction to detect the labeled compound.
